(19) United States
(12) Patent Application Publication
(10) Pub. No.: US 2008/0213248 A1
(43) Pub. Date: Sep. 4, 2008

(54) Methods and Systems for Treating Asthma and Other Respiratory Diseases

(75) Inventor: Jianguo G. Gu, Gainesville, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: UNIVERSITY OF FLORIDA RESEARCH FOUNDATION, INC., Gainsville, FL (US)

(21) Appl. No.: 11/661,666

(22) PCT Filed: Sep. 2, 2005
(86) PCT No.: PCT/US05/31686

§ 371 Date: May 26, 2008

Related U.S. Application Data
(60) Provisional application No. 60/607,006, filed on Sep. 2, 2004.

(51) Int. Cl. A61K 39/395; A61K 38/02; A61K 48/00; A61K 31/497; A61K 31/55; A61K 36/61; A61K 36/534; A61K 31/045; A61K 31/22; A61K 31/4015; A61K 31/352; A61P 11/00; G01N 33/53

(52) U.S. Cl. 424/130.1; 514/2; 514/44; 514/255.05; 514/213.01; 424/742; 424/747; 514/729; 514/546; 514/424; 514/456; 514/739; 435/7.1

(57) ABSTRACT

The subject invention provides methods for using TRPM8 receptors as a therapeutic target for identifying effective methods or compounds useful in the diagnosis or treatment of respiratory diseases or conditions. In certain embodiments, compositions that are identified using the methods of the invention are administered to a patient to diagnose or treat a respiratory disease or condition. In a preferred embodiment, TRPM8 receptor blockers are identified and administered, in accordance with the subject invention, to treat a patient with a respiratory disease, such as asthma.

### CROSS-REFERENCE TO RELATED APPLICATION

The present application claims benefit of U.S. Provisional Application Ser. No. 60/607,006, filed Sep. 2, 2004, which is hereby incorporated by reference herein in its entirety, including any figures, tables, nucleic acid sequences, amino acid sequences, and drawings.

### GOVERNMENT SUPPORT

The subject matter of this application has been supported by a research grant from the National Science Foundation (Grant Number NSF: 0237317). Accordingly, the government may have certain rights in this invention.

### BACKGROUND OF THE INVENTION

Respiratory diseases such as asthma are reaching epidemic proportions in both developed and under-developed countries (Holt, P. G. et al., “The role of allergy in the development of asthma,” Nature, 402 (6760 Supplement):B12-7 (1999)). Such diseases, and asthma in particular, cause distress and misery in millions, often at a time in their lives when they should be most active. For example, asthma interferes with sleep, intellectual functioning and recreational activities. At one extreme, asthma can be life-threatening, and can cause deaths that are often avoidable.

In Britain and Australia, 1 in 4 children under the age of 14 years as have asthma. In the United States, approximately fifteen million individuals have asthma and the disease is the cause of more than five thousand deaths annually in the U.S. In children, asthma is the most prevalent chronic disease, requiring the most frequent use of emergency room visits and hospitalizations. The overall annual cost for asthma care in the U.S. is estimated to be in the range of billions of dollars. Asthma is the most common cause of school and work absenteeism in the U.S.

Respiration is a key component of human life. The lungs remove oxygen from air for transport via the blood stream to the entire body. Entrance of air to the lungs must travel through bronchial tubes, which can open or close in response to many stimuli. For example, once bronchi constrict and plug with mucus in response to inhaled allergens, as occurs in asthma, the quantity of air is greatly impaired and oxygen starvation begins. Continual evolution of a constricted and mucus filled bronchial tree, such as those of asthmatics, is always life threatening.

Control of normal breathing is largely under the direction of the brain stem. However, part of the limbic system of the brain and hypothalamus has the ability to accelerate the pattern of breathing in times of fear or rage. There are chemoreceptors involved in minute-by-minute breathing control which are located in the vicinity of the exit points of the ninth (glossopharyngeal) and tenth (vagus) nerves of the medulla oblongata, near the medulla oblongata's ventral surface.

The central nervous system nerves involved in breathing are the second, third, fourth, fifth, eighth, ninth, and the important tenth (vagus). The first cranial nerve supplies olfactory information and the second and third nerves are related to inputs from the eyes as afferent sensors which integrate what the body is perceiving from outside and demands faster or slower breathing rates or even holding ones breath. The eighth cranial nerve provides auditory afferent input. The various afferent sensory systems provide information as to how the body should be breathing in response to events outside the body proper.

Recently, molecular mechanisms for sensing of cold by the primary somatic sensory afferent nerve endings of the skin have been studied by electrophysiological approaches and molecular cloning methods. Cool temperatures excite a subpopulation of primary somatic sensory afferent nerves by opening a cation channels on the cell membranes of the somatic sensory afferent fibers, which cause action potential firing on cool-sensing somatic afferent fibers (Okazawa et al., “Ionic basis of cold receptors acting as thermostats,” J. Neurosci., 22 (10):3994-4001 (2002); Reid et al, “Ion channels activated by cold and menthol in cultured rat dorsal root ganglion neurones,” Neurosci. Lett., 324 (2):164-168 (2002)). In addition to cool temperatures, several cooling compounds such as menthol can also open the same cation channels, producing a cooling sensation. The molecular substrate of cool temperature transduction at skin nerve endings becomes clear with the recent cloning of a cool temperature receptor called cold and menthol receptor-1 (CMR1, McKemy et al., “Identification of a cold receptor reveals a general role for TRP channels in thermosensation,” Nature, 416 (6876):52-8 (2002)).

CMR1 receptor is also called transient receptor potential channel M8 (TRPM8, Peier at al., “A TRP channel that senses cold stimuli and menthol,” Cell, 108 (5):705-15 (2002)). Similar to capsaicin VR1 receptors (noxious heat receptors) and other thermoreceptors, TRPM8 receptor belongs to the transient receptor potential (TRP) super-family (Clapham et al., “The TRP ion channel family,” Nat Rev Neurosci., 2 (6):387-96 (2001); Minke and Cook, “TRP channel proteins and signal transduction,” Physiol Rev., 82 (2):429-72 (2002)). Electrophysiological studies have indicated that TRPM8 is a ligand-gated cation channel highly permeable to Ca²⁺ (McKemy et al., ibid.; Peier at al., ibid.; Reid et al., ibid.), and activation of TRPM8 can result in a large increase of intracellular Ca²⁺ levels (Okazawa et al., ibid.; McKemy et al., ibid.; Peier et al., ibid.; Reid et al., ibid., Tsukuki et al., “Menthol-induced Ca²⁺ release from presynaptic Ca²⁺ stores potentiates sensory synaptic transmission,” J. Neurosci., 24 (3):762-71 (2004)) through both Ca²⁺ entry from extracellular sites and Ca²⁺ release from intracellular stores (Tsuzuski et al., ibid.).

When expressed on heterologous cell system, cool temperatures below 24° C. to 28° C. can evoke robust membrane currents through TRPM8. TRPM8 currents increase with decreases of temperatures and reach maximum currents near 10° C. Current elicited by cold temperatures in heterologous cells expressing TRPM8 have biophysical properties indistinguishable from those observed in cool-sensitive somatic afferent neurons under similar conditions (McKemy et al., ibid.). Thus, TRPM8 is believed to serve as a sensor of cool temperatures at peripheral nerve endings innervating the skin (McKemy et al., ibid.; Peier at al., ibid.). TRPM8 can also be activated by menthol, an active ingredient of peppermint that produces a cooling sensation (McKemy et al., ibid.; Peier at al., ibid.; Reid et al., ibid.). In addition, several other chilling compounds can activate TRPM8 (McKemy et al., ibid.).

The vagus afferent nerves and the somatic sensory afferent fibers are two nervous systems that are not only anatomically, but also functionally, different. Functionally, the somatic sensory afferent fibers sense stimuli and produce a conscious sensation. On the other hand, stimulation of vagus afferent nerves only produce autonomic reflex without having conscious sensation. Little is known, to date, regarding the autonomic function of TRPM8 receptors in vagus afferent nerves for bronchopulmonary areas.

What is currently known is that respiratory control is activated by the vagus nerve and its preganglionic nerve fibers, which synapse in ganglia embedded in the bronchi that are also enervated with sympathetic and parasympathetic activity. The sympathetic nerve division can have no effect on bronchi or it can dilate the lumen (bore) to allow more air to enter the respiratory process, which is helpful to asthma patients, while the parasympathetic process offers the opposite effect and is able to constrict the bronchi and increase secretions, which is harmful to asthma patients.

The mechanisms responsible for the development of asthma in atopic patients include genetic predisposition and the effects of environmental exposures to inflammatory stimuli in the airways of susceptible individuals (Bleecker, E. R., and D. A. Meyers in Genetics of Allergy and Asthma. M. N. Blumenthal, and B. Bjorksten, (eds. Marcel Dekker, New York, p. 307 (1997)). For example, it is known that cold temperature exacerbates respiratory disorders, including asthma (Koskela, H. O. et al., “Responsiveness to three bronchial provocation tests in patients with asthma,” Chest., 124 (6):2171-7 (2003)). Inhalation of cold air is also a cause of asthma attack in athletes performing winter sports (Weiler, J. M. and Ryan, E. J. 3^{rd}, “Asthma in United States olympic athletes who participated in the 1998 olympic winter games,” J Allergy Clin Immunol., 106 (2):267-71 (2000)). Clinically, cold air challenge has been routinely used in the diagnosis of asthma (Galdes-Sebaldt, M. et al., “Comparison of cold air, ultrasonic mist, and methacholine inhalations as tests of bronchial reactivity in normal and asthmatic children,” J. Pedatr., 107 (4):526-30 (1985)).

Bronchial asthma in mammals is characterized by inflammation of the airways, exaggerated airway reactivity to bronchoconstrictor agonists, and attenuated β-adrenoceptor-mediated airway relaxation (Bai, T. R., “Abnormalities in airway smooth muscle in fatal asthma,” Am. Rev. Respir. Dis., 141:552-557 (1990); Goldie, R. G. et al., “In vitro responsiveness of human asthmatic bronchus to carbachol, histamine, beta-adrenoceptor agonists and theophylline,” Br. J. Clin. Pharmacol., 22:669-676 (1986); McFadden, E. R., Jr., “Asthma: morphologic-physiologic interactions,” Am. J. Respir. Crit. Care Med., 150 (5 Pt 2):S23-6 (1994)). Unfortunately, little is known, at the receptor level, how cold temperature results in respiratory disorders, including asthma.

Current treatment options for asthma include medications that control the airway inflammatory component of the disease, e.g., primarily corticosteroids, sodium cromolyn, methylxanthines, leukotriene modifiers) and rapid relief medications that counteract bronchospasm, e.g., primarily beta-adrenergic agents. There are several disadvantages to using these medications as follows. There is a potential lack of effective sustained action; there are side effects associated with prolonged use of these medications, particularly in the case of corticosteroids and beta-adrenergic agents; there is a progressive loss of sensitivity to these treatments after prolonged use; there is limited efficacy of any of these agents in severe cases of asthma; these agents are non-selective, i.e., they do not specifically target the lung, therefore, side-effects affecting other organs are a potential risk. Furthermore, documented data suggests an increased risk of dying from bronchial asthma following prolonged treatment of asthma using long-acting beta-adrenergic agents such as fenoterol (Pearce, N. et al., “Case-control study of prescribed fenoterol and death from asthma in New Zealand, 1977-81,” Thorax., 45:170-175 (1990); Spitzer, W. O. et al., “The use of beta-agonists and the risk of death and near death from asthma,” N. Engl. J. Med., 326:560-561 (1992)).

There is thus a long felt need for additional and more specific and effective compositions and methods for treatment of asthma and other respiratory diseases, which overcome the deficiencies of the prior art compositions and methods.

### BRIEF SUMMARY

According to the subject invention, systems and methods are provided for effective diagnosis and treatment of respiratory diseases, including asthma. In one embodiment, the invention comprises the use TRPM8 receptors as targets for screening compounds or methods in order to identify compounds and/or methods useful in the diagnosis or treatment of asthma and other respiratory diseases. In certain embodiments, the compounds identified by the screening methods of the invention are administered to a patient to diagnose and/or treat asthma and other respiratory diseases.

As described above, recent studies regarding TRPM8 receptors and other sensory molecules have focused on their function in somatic sensory systems, not on vagus autonomic nervous system. The subject invention provides methods for identifying TRPM8 receptors that are involved in cold-induced vagus nerve (autonomic) reflex of the bronchopulmonary system. The bronchopulmonary afferent fibers of the vagus nerve are autonomic nerves that control airway resistance and mucosa secretion through central and local neuronal reflex, and play important roles in respiratory pathology conditions including asthma. Such methods enable the user to better understand the molecular mechanism of autonomic respiratory response to cold and other chilling compounds. More importantly, the subject invention advantageously demonstrates vagus nerve response to cold and menthol through the activation of TRPM8 receptors.

Accordingly, the subject invention is directed to the use of TRPM8 receptors, or its fragments, as a therapeutic target for identifying effective compositions or methods useful in the diagnosis and/or treatment of respiratory diseases or conditions.

In preferred embodiments of the invention, natural or synthetic peptides, ligands, blockers, agonists, antagonists, inhibitors, antibodies, polynucleotides, and modulators that can bind to TRPM8 receptors are used in the diagnosis and/or treatment of respiratory diseases, including asthma. More preferably, TRMP8 receptor blockers, such as 2-aminoethoxydiphenylborane and compounds developed therefrom, can be tested and/or administered to a patient to treat a respiratory disease or condition, such as asthma.

According to the subject invention, any known technique used for biological screening and characterization of potential drugs can be used on TRPM8 receptors, or its fragments, to screen for compounds and/or methods that effectively treat and/or diagnose respiratory diseases or conditions.

In one embodiment, compounds that are identified by the subject invention as effective in regulating TRPM8 receptor activity (i.e., agonists, antagonists, blockers, modulators) are employed as part of a pharmaceutical composition for use in diagnosing or treating a respiratory disease or condition. For example, compounds that are identified as TRPM8 receptor agonists can be employed as a part of a pharmaceutical composition for use in diagnosing asthma. In another example, pharmaceutical compositions may be formulated using TRPM8 receptor blockers to treat asthma.

In another embodiment, gene therapy that manipulates TRPM8 receptor activity can be administered to a patient diagnosed with a respiratory disease or condition. In certain embodiments of the invention, TRPM8 receptor nucleic acid sequences are incorporated into effective expression vectors and directly administered into cells for gene therapy. In like manner, RNA transcripts produced in vitro may be encapsulated in and administered via liposomes. Such vectors and/or transcripts can function transiently or may be incorporated into the host chromosomal DNA for longer term expression. For example, gene transfer of antisense RNA sequence using viral vector systems to silence TRPM8 receptor activity can be used in accordance with the subject invention to treat a respiratory disease or condition.

In vivo delivery of genetic constructs into patients is developed to the point of targeting specific cell types. The delivery of specific cells has been accomplished, for instance, by complexing nucleic acids with proteinous ligands that recognize cell specific receptors that mediate uptake (see, for example, Wu, G. Y. et al. (1991) J. Biol. Chem., 266:14338-42). Alternatively, recombinant nucleic acid constructs may be injected directly for local uptake and integration (see, for example, Jiao, S. et al. (1992) Human Gene Therapy, 3:21-33).

### BRIEF DESCRIPTION OF THE DRAWINGS

FIG. 1a is a diagram illustrating the instillation of a tracing dye into the lower segment of rat trachea to retrograde label airway vagus ganglion neurons.

FIG. 1b is a micrograph that illustrates a field of acutely dissociated vagus ganglion neurons under bright light and fluorescence light.

FIG. 1c illustrates images of increases in [Ca²⁺]ᵢₙ in labeled vagus ganglion neurons when exposed t various conditions, including menthol and cold temperature.

FIGS. 1d through 1g illustrate time course responses in labeled vagus ganglion neurons to various conditions.

FIGS. 2a and 2b are graphical illustrations of results provided from a set of experiments using calcium-imaging technique to demonstrate cold temperature and cooling compounds stimulation of bronchopulmonary afferent fibers of vagus nerve.

FIGS. 3a and 3b illustrate the expression of TRPM8 receptors on the ganglion neurons of vagus nerve using immunochemistry with an antibody against TRPM8 receptors.

FIGS. 4a through 4c illustrate various images of TRPM8 immunoreactivity in various locations.

FIGS. 5a through 5d illustrate TRPM8 receptor activation on the vagus nerve using patch-clamp electrophysiology technique.

FIG. 6a illustrates micrographs illustrating labeled vagus ganglion neuron and subsequent patched-clamp recording.

FIGS. 6b and 6c illustrate sample traces of whole-cell inward currents evoked from vagus ganglion neuron when subjected to various conditions in accordance with the subject invention.

FIG. 6d illustrates the pooled results from experimental results illustrated in FIGS. 6B and 6C.

FIGS. 6e and 6f illustrate the I-V relationship of menthol evoked currents from a vagus ganglion neuron as opposed to dorsal root ganglion neurons.

FIG. 6g illustrates a graphical comparison of menthol-evoked currents in vagus ganglion neurons and dorsal ganglion neurons.

FIGS. 6h and 6i illustrate action potentials in the vagus ganglion neurons when subjected to various conditions in accordance with the subject invention.

FIGS. 7a through 7e illustrate fluorescence intensity in a retrograde labeled vagus ganglion neuron of the invention.

FIG. 7f is a micrograph of a retrograde labeled vagus ganglion neuron of the invention.

FIGS. 7g through 7i illustrate fluorescence intensity changes over time in the cells shown in FIGS. 7b, 7c, and 7d.

FIG. 8a illustrates the number of TRPM8-expressing neurons in cell culture in the presence of nerve growth factor.

FIG. 8b illustrates TRPM8 expressing neuron response to the agonist menthol in different cultures.

### DETAILED DISCLOSURE

The present invention is broadly directed to methods for screening compounds or methods useful in the diagnosis or treatment of respiratory diseases or conditions as well as the diagnostic/therapeutic compositions or methods derived therefrom. In particular, the TRPM8 receptor is used as a therapeutic target for screening compounds or methods useful in the treatment of respiratory diseases or conditions. Thus, the subject invention gives rise to compositions or methods useful in the diagnosis or treatment of respiratory diseases or conditions.

In one embodiment, the invention is based on the use of known drug screening methods for identifying compounds that bind to and modulate TRPM8 receptor activity, in particular TRPM8 receptor activity involved in cold-induced vagus nerve reflex. According to the subject invention, screening techniques including, but not limited to, microphysiometry, immunoassays, combinatorial libraries, separation techniques coupled to mass spectrometry, enzyme assays using proteases, molecular imprinting techniques, aptamer-based screening systems, high-throughput screening methods, and electrophysiological screening methods, are useful in identifying effective compounds or methods that regulate TRPM8 receptor activity. Information regarding the identified compounds or methods is employed to: 1) treat respiratory diseases or conditions; or 2) diagnose respiratory diseases or conditions.

### DEFINITIONS

Unless otherwise stated, the following terms used in the specification and claims have the meanings given below.

The term “treat” or any derivative thereof (i.e., treatment, treating), as used herein, refers to and includes: 1) preventing a respiratory disease or condition from occurring in a patient that may be predisposed to the respiratory disease or condition but has not yet been diagnosed as having it; 2) inhibiting the respiratory disease or condition, e.g., arresting its development; or 3) relieving the respiratory disease or condition, e.g., causing regression of the disease.

As used herein, the term “asthma” refers to a respiratory disorder characterized by episodic difficulty in breathing brought on by any one or a combination of three primary factors including: 1) bronchospasm (i.e., variable and reversible airway obstruction due to airway muscle contraction), 2) inflammation of the airway lining, and 3) bronchial hyperresponsiveness resulting in excessive mucous in the airways, which may be triggered by exposure to an allergen or combination of allergens (i.e., dust mites and mold), viral or bacterial infection (i.e., common cold virus), environmental pollutants (i.e., chemical fumes or smoke), physical over exertion (i.e., during exercise), stress, or inhalation of cold air.

The term “asthmatic condition,” as used herein, refers to the characteristic of an individual to suffer from an attack of asthma upon exposure to any one or a number of asthma triggers for that individual. An individual may be characterized as suffering from, for example, allergen-induced asthma, exercise-induced asthma, pollution-induced asthma, viral-induced asthma, or cold-induced asthma.

The terms “chronic obstructive pulmonary disease” and “COPD” as used interchangeably herein refers to a chronic disorder or combination of disorders characterized by reduced maximal expiratory flow and slow forced emptying of the lungs that does not change markedly over several months and is not, or is only minimally, reversible with traditional bronchodilators. Most commonly, COPD is a combination of chronic bronchitis, i.e. the presence of cough and sputum for more than three months for about two consecutive years, and emphysema, i.e. alveolar damage. However, COPD can involve chronic bronchitis with normal airflow, chronic bronchitis with airway obstruction (chronic obstructive bronchitis), emphysema, asthmatic bronchitis, and bullous disease, and combinations thereof.

The term “respiratory” refers to the process by which oxygen is taken into the body and carbon dioxide is discharged, through the bodily system including the nose, throat, larynx, trachea, bronchi and lungs.

The term “respiratory disease or condition” refers to any one of several ailments that involve inflammation and affect a component of the respiratory system including especially the trachea, bronchi and lungs. Such ailments include asthmatic conditions including allergen-induced asthma, exercise-induced asthma, pollution-induced asthma, cold-induced asthma, stress-induced asthma and viral-induced-asthma, chronic obstructive pulmonary diseases including chronic bronchitis with normal airflow, chronic bronchitis with airway obstruction (chronic obstructive bronchitis), emphysema, asthmatic bronchitis, and bullous disease, and other pulmonary diseases involving inflammation including cystic fibrosis, pigeon fancier's disease, farmer's lung, acute respiratory distress syndrome, pneumonia, aspiration or inhalation injury, fat embolism in the lung, acidosis inflammation of the lung, acute pulmonary edema, acute mountain sickness, post-cardiac surgery, acute pulmonary hypertension, persistent pulmonary hypertension of the newborn, perinatal aspiration syndrome, hyaline membrane disease, acute pulmonary thromboembolism, heparin-protamine reactions, sepsis, status asthamticus and hypoxia.

A “patient,” as used herein, describes an organism, including mammals, from which bodily fluid samples are collected and for which diagnosis and/or treatment is provided in accordance with the present invention. Mammalian species that benefit from the disclosed systems and methods of diagnosis include, and are not limited to, apes, chimpanzees, orangutans, humans, monkeys; and domesticated animals (e.g., pets) such as dogs, cats, mice, rats, guinea pigs, and hamsters.

As used herein, the term “agonist” refers to a molecule that, when bound to a TRPM8 receptor, causes a change in TRPM8 receptor that modulates the activity of the TRPM8 receptor. Agonists may include proteins, nucleic acids, carbohydrates, or any other molecules that bind to TRPM8 receptors.

The term “antagonist” or “inhibitor,” as used herein, refers to a molecule that, when bound to a TRPM8 receptor, blocks or modulates the biological activity of the TRPM8 receptor. Antagonists and inhibitors may include proteins, nucleic acids, carbohydrates, or any other molecules that bind to the TRPM8 receptor.

The term “modulate,” as used herein, refers to a change or an alteration in the biological activity of TRPM8 receptors. Modulation may be an increase or a decrease in protein activity, a change in binding characteristics, or any other change in the biological or functional properties of TRPM8 receptors.

The term “antisense,” as used herein, refers to nucleotide sequences that are complementary to a specific DNA or RNA sequence. Antisense molecules can be produced by any method, including synthesis by ligating the gene(s) of interest in a reverse orientation to a viral promoter that permits the synthesis of a complementary strand. Once introduced into a cell, this transcribed strand combines with natural sequences produced by the cell to form duplexes. These duplexes then block either further transcription or translation.

Using the screening methods of the invention, useful methods or compounds are identified that can be utilized for diagnostic and/or therapeutic purposes in treating various respiratory diseases or conditions. The present invention has identified TRPM8 receptors, or fragments thereof, as a therapeutic target for the diagnosis and/or treatment of respiratory diseases and conditions where, as provided by this invention, TRPM8 receptors mediate bronchopulmonary afferent fiber response to cold temperature and cooling compounds. The TRPM8 receptors, as described herein (i.e., newly associated with mediation of bronchopulmonary afferent fiber response), provide a new target for diagnostic methods and drug discovery and treatments of respiratory diseases or conditions. Therapeutic methods identified by the subject invention include, but are not limited to, gene therapy. Therapeutic compounds identified by the subject invention include, but are not limited to, natural or synthetic peptides, ligands, blockers, agonists, antagonists, inhibitors, antibodies, polynucleotides, and modulators.

According to the subject invention, contemplated TRPM8 receptor agonists include, but are not limited to, cooling agents (i.e., menthol; eucalyptus oil; peppermint oil; icilin; cyclohexanol, 5-methyl-2-(1-methylethenyl)-, available from Takasago International Corporation, Tokyo under the tradename, COOLACT; 6-Isopropyl-9-methyl-1,4-dioxaspiro-(4,5)decane-2-methanol, (I)-menthone glycerol ketal (Menthone Glycerin Acetal) available from Haarmann & Reimer (“H&R”) under the tradename, FRESCOLAT MGA; 5-methyl-2-(1-methyl ethyl)-cyclohexyl-2-hydroxypropionate, 1-menthyl lactate, acid/-menthyl ester (Menthyl Lactate) available from H&R under the tradename, FRESCOLAT ML; menthyl pyrrolidone carboxylate (Menthyl PCA) available from Quest International UK Limited under the tradename, QUESTICE), eucalyptol, linalool, geraniol, hydroxycitronellal, WS-3 (novel RGD-containing protein), WS-23, and mixtures thereof.

Contemplated TRPM8 receptor antagonists or blockers of the invention include, but are not limited to, N-(4-tertiarybutylphenyl)-4-(3-chloropyridin-2-yl)tetrahydropyrazine-1(2H)-carbox-amide (BCTC), thio-BCTC, capsazepine, and 2-aminoethoxydiphenylborane. In a preferred embodiment, pharmaceutical compositions that include 2-aminoethoxydiphenylborane are administered to a patient to treat respiratory diseases or conditions, in particular, asthma.

### Screening Methods

### 1. Ligand-Binding Assays

The specificity of molecular recognition of certain compounds (i.e., antigens by antibodies) to form a stable complex is the basis of both the analytical immunoassay in solution and the immunosensor on solid-state interfaces. The underlying fundamental concept of these analytical methods as ligand-binding assays is based on the observation of the products of the ligand-binding reaction between the target analyte (i.e., a compound that binds to TRPM8 receptors) and a highly specific binding reagent.

In certain instances, alternative analyte-binding compounds such as aptamers are applied to ligand-binding assays for particularly sensitive screening. Aptamers are single-stranded DNA or RNA oligonucleotide sequences with the capacity to recognize various target molecules with high affinity and specificity. These ligand-binding oligonucleotides mimic properties of antibodies in a variety of diagnostic formats. They are folded into unique overall shapes to form intricate binding furrows for the target structure. Aptamers are identified by an in vitro selection process known as systematic evolution of ligands by exponential enrichment (SELEX). Aptamers may have advantages over antibodies in the ease of depositing them on sensing surfaces. Moreover, due to the highly reproducible synthetic approach in any quantities, albeit the affinity constants are consistently lower than those of antibodies and the stability of these compounds is still questionable, they may be particularly useful for diagnostic applications in complex biological matrices (i.e., in diagnosing patients with respiratory diseases or conditions). The aptamer-based schemes are still in their infancy and it is expected that modified nuclease-resistant RNA and DNA aptamers will soon be available for a variety of therapeutic and diagnostic formats.

The potential of aptamers for use in biosensors has been outlined in the design of a fiber-optic biosensor using an anti-thrombin DNA aptamer, immobilized on the surface of silica microspheres and distributed into microwells on the distal tip of the imaging fiber. With this device, the determination of thrombin at low concentration was possible. Exciting new possibilities are evolving by the introduction of signaling aptamers with ligand-dependent changes in signaling characteristics and catalytically active so-called “apta-zymes” which would allow the direct transduction of molecular recognition to catalysis.

Another alternative analyte-binding compound for use in immunoassays is the anticalin. Lipocalins constitute a family of proteins for storage or transport of hydrophobic and/or chemically sensitive organic compounds. The retinol-binding protein is an example in human physiology. It has been demonstrated that the bilin-binding protein, a member of the lipocalin family and originating from the butterfly Pieris brassicae, can be structurally reshaped in order to specifically complex potential antigens, such as digoxigenin, which was given as an example. These binding proteins share a conserved β-barrel, which is made of eight antiparallel β-strands, winding around a central core. At the wider end of the conical structure, these strands are connected in a pairwise manner by four loops that form the ligand binding site. The lipocalin scaffold can be employed for the construction of so-called “anticalins”, which provide a promising alternative to recombinant antibody fragments. This is made by individualizing various amino acid residues, distributed across the four loops, to targeted random mutagenesis. It remains to be shown that this class of proteins is applicable in diagnostic assays and in immunosensors. Critical points that still need to be defined include the synthesis and stability of the anticalins, the magnitude of the affinity constants, and the versatility for being crafted against the large variety of ligands.

Alternatively, molecular imprinting techniques may be used to screen compounds or methods that affect TRPM8 receptor activity. This is a technique that is based on the preparation of polymeric sorbents that are selectivity predetermined for a particular substance, or group of structural analogs. Functional and cross-linking monomers of plastic materials, such as methacrylics and styrenes, are allowed to interact with a templating ligand to create low-energy interactions. Subsequently, polymerization is induced. During this process, the molecule of interest is entrapped within the polymer either by a noncovalent, self-assembling approach, or by a reversible, covalent approach. After stopping the polymerization, the template molecule is washed out. The resultant imprint of the template is maintained in the rigid polymer and possesses a steric (size, shape) and chemical (special arrangement of complementary functionality) memory for the template. The molecularly imprinted polymer (MIP) can bind the template (=analyte) with a specificity similar to that of the antigen-antibody interaction.

Besides the main applications in solid-phase extraction and chromatography, molecularly imprinted polymers have already been employed as nonbiological alternatives to antibodies in competitive binding assays. A series of applications for analytes, such as cyclosporin A1 atrazine, cortisol, 17b-estradiol, theophylline, diazepam, morphine, and S-propranolol, suggests that molecular imprinting is a promising technique for immunoassays.

A biosensor is an analytical device that integrates a biological element on a solid-state surface, enabling a reversible biospecific interaction with the analyte, and a signal transducer. The biological element is a layer of molecules qualified for biorecognition, such as enzymes, receptors, peptides, single-stranded DNA, or even living cells. Compared to conventional analytical instruments, biosensors are characterized by an integrated structure of these two components. Many devices are connected with a flow-through cell, enabling a flow-injection analysis (FIA) mode of operation. Biosensors combine high analytical specificity with the processing power of modern electronics to achieve highly sensitive detection systems.

There are two different types of biosensors: biocatalytic and bioaffinity-based biosensors. The biocatalytic biosensor uses mainly enzymes as the biological compound, catalyzing a signaling biochemical reaction. The bioaffinity-based biosensor, designed to monitor the binding event itself, uses specific binding proteins, lectins, receptors, nucleic acids, membranes, whole cells, antibodies or antibody-related substances for biomolecular recognition.

### 2. Microphysiometry

During the growth of a typical biological cell, carbon-containing nutrients such as glucose are taken up and acidic metabolic products such as lactic acid are released. In microphysiometry, these changes in metabolic rate are recorded as changes in the rate of acidification of the medium surrounding the cells (i.e., Raley-Susman, K. M. et al., “Effects of excitotoxin exposure on metabolic rate of primary hippocampal cultures: application of silicon microphysiometry to neurobiology,” J. Neurosci., 12 (3):773-780 (1992); Baxter, G. T. et al., “PKC_{ε} is involved in granulocyte-macrophage colony-stimulating factor signal transduction: evidence from microphysiometry and antisense oligonucleotide experiments,” Biochemistry, 31:19050-10954 (1992); Bouvier, C. et al., “Dopaminergic activity measured in D₁- and D₂-transfected fibroblasts by silicon microphysiometry,” J. Recept. Res., 13 (1-4):559-571 (1993); and McConnell, H. M. et al., “The cytosensor microphysiometer: biological applications of silicon technology,” Science, 257:1906-1912 (1992)).

Virtually any molecule that affects the cell can be detected using microphysiometry. Such molecules include neurotransmitters, growth factors, cytokins, receptors, and the like. Thus, the microphysiometry method can provide valuable information regarding compounds that affect TRPM8 receptor activity.

### 3. Use of Combinatorial Libraries

Synthetic combinatorial libraries have proven to be a valuable source of diverse structures useful for large-scale biochemical screening (i.e., Sastry, L. et al., “Screening combinatorial antibody libraries for catalytic acyl transfer reactions,” Ciba Found Symp., 159:145-155 (1991); Persson, M. A. A. et al., “Generation of diverse high-affinity monoclonal antibodies by repertoire cloning,” Proc. Natl. Acad. Sci. USA, 33:2432-2436 (1991); and Houghten, R. A., “Generation and use of synthetic peptide combinatorial libraries for basic research and drug discovery,” Nature, 354:84-86 (1991)). Such libraries are generated by a combination of solution and solid-phase chemistries and are cleaved off the solid-support for screening.

### 4. Separation Techniques Coupled to Mass Spectrometry

Separation techniques such as liquid chromatography, gas chromatography, and capillary electrophoresis coupled to mass spectrometry or tandem-mass spectrometry create analytical systems available for structural evaluation (i.e., Hsieh, S. et al., “Separation and identification of peptides in single neurons by microcolumn liquid chromatography—Matrix-assisted laser desorption/ionization time-of-flight mass spectrometry and postsource decay analysis,” Anal Chem., 70 (9):1847-1852 (1998); Tretyakova, N. Y. et al., “Quantitative analysis of 1,3-butadene-induced DNA adducts in vivo and in vitro using liquid chromatography electrospray ionization tandem mass spectrometry,” J. Mass Spectrom., 33:363-376 (1998); and Taylor, G. W. et al., “Excursions in biomedical mass spectrometry,” Br. J. Clin. Pharmacol., 41:119-126 (1996)). Mass spectrometry is particularly useful in providing information about the molecular weight of a compound/molecule. With refined and controlled fragmentation of large molecules, it is also possible to extraction information about the sequence.

### 5. High-Throughput Screening

High-throughput screening measures intracellular calcium fluxes. Calcium ions are widely used in nature as intracellular messengers for mediating signal transduction, wherein the binding of a signal molecule to an extracellular receptor triggers a sequence of events leading to the release of calcium from the endoplasmic reticulum.

One high-throughput screening method utilizes fluorescent indicators that selectively respond to Ca²⁺. Indicators such as visible-light-excitable fluo-3 and fluo-4, as disclosed in U.S. Pat. Nos. 5,516,911 and 5,049,673 and European Patent 0 314 480, can be used to demonstrate spatial and temporal localization of calcium responses within cells. Such indicators often exhibit a dramatic increase in fluorescence intensity upon calcium binding.

Another high-throughput screening method utilizes light excitable indicators. Examples of ultra-violet-light-excitable indicators include fura-2 and indo-1 indicators, as disclosed in U.S. Pat. No. 4,603,209, which undergo a shift in excitation or emission spectra upon binding to calcium. Such spectral changes can be exploited to provide valuable quantitative information about intracellular calcium levels.

Further high-throughput screening methods utilize dyes that are sensitive to membrane potential changes due to ion flux through cell membranes. The ability to measure ion fluxes is not only useful for pharmacological characterization (i.e., whether an agonist can activate a receptor or an antagonist can block a receptor) but also for screening new surrogate ligands. For example, voltage sensor dyes or “probes” (a Fluorescence Resonance Energy Transfer (FRET)-based voltage sensing assay technology) have been disclosed for use in measuring changes in cellular membrane electrical potential.

### 6. Electrophysiology Screening

Voltage/patch clamp methods are common techniques used to investigate the properties of excitable cells (i.e., nerve and muscle cells). Such methods are particularly useful in revealing the role of channels and ion-channel function. Specifically, a voltage-clamp method is a method for maintaining the voltage inside a cell (the membrane potential) at a constant value and, at the same time, measuring the flow of current across the membrane. The current is generated by charged ions (i.e., Na⁺, K⁺, Ca⁺, Cl⁻) moving through ion channels in the surface membrane. Electrodes are used to provide and measure the amount of current flowing at a particular voltage (i.e., very small changes in membrane potential). The voltage/patch clamp method is particularly useful when used to study current flow through a particular type of ion channel.

### Diagnostic Use

Particular antibodies for TRPM8 receptors are useful for the diagnosis of conditions or diseases characterized by expression of TRPM8 receptors or in assays to monitor patients being treated with TRPM8 receptor modulating compounds (such as agonists or inhibitors). Diagnostic assays for TRPM8 receptors include methods utilizing particular antibodies and a label to detect TRPM8 receptors in patient (such as human) body fluids or extracts of cells or tissues. The polypeptides and antibodies of the present invention may be used with or without modification.

TRPM8 receptor antibodies useful for the diagnosis of respiratory diseases and/or conditions associated with TRPM8 receptor activity include, but are not limited to, polyclonal, monoclonal, chimeric, single chain, Fab fragments, and fragments produced by a Fab expression library. Neutralizing antibodies, such as those that inhibit dimer formation, are especially preferred for diagnostics and therapeutics.

Monoclonal antibodies to TRPM8 receptors can be prepared using any technique that provides for the production of antibody molecules by continuous cell lines in culture. These include, but are not limited to, the hybridoma technique originally described by Koehler and Milstein (1975, Nature, 256:495-497), the human B-cell hybridoma technique (Kosbor et al. (1983) Immunol Today, 4:72; Cote et al. (1983) Proc Natl Acad Sci, 80:2026-2030) and the EBV-hybridoma technique (Cole et al. (1985) Monoclonal Antibodies and Cancer Therapy, Alan R Liss Inc., New York, N.Y., pp. 77-96).

In addition, techniques developed for the production of “chimeric antibodies,” the splicing of mouse genes to human antibody genes to obtain a molecule with appropriate antigen specificity and biological activity can be used (see Morrison et al. (1984) Proc Natl Acad Sci, 81:6851-6855; Neuberger et al. (1984) Nature, 312:604-608; Takeda et al. (1985) Nature, 314:452-454). Alternatively, techniques described for the production of single chain antibodies (see, for example, U.S. Pat. No. 4,946,778) can be adapted to produce TRPM8 receptor specific single chain antibodies.

Antibodies may also be produced by inducing in vivo production in the lymphocyte population or by screening recombinant immunoglobulin libraries or panels of highly specific binding reagents as disclosed in Orlandi et al. (1989, Proc Natl Acad Sci. 86: 3833-3837), and Winter G. and Milstein C. (1991; Nature 349:293-299).

Antibody fragments which contain specific binding sites for TRPM8 receptors can also be generated. For example, such fragments include, but are not limited to, the F(ab′)2 fragments which can be produced by pepsin digestion of the antibody molecule and the Fab fragments which can be generated by reducing the disulfide bridges of the F(ab′)2 fragments. Alternatively, Fab expression libraries may be constructed to allow rapid and easy identification of monoclonal Fab fragments with the desired specificity (see, for example, Huse W D et al (1989) Science 256:1275-1281).

A variety of protocols for competitive binding or immunoradiometric assays using either polyclonal or monoclonal antibodies with established specificities are well known in the art. Such immunoassays typically involve the formation of complexes between SURH and its specific antibody and the measurement of complex formation. A two-site, monoclonal-based immunoassay utilizing monoclonal antibodies reactive to two noninterfering epitopes on a specific SURH protein is preferred, but a competitive binding assay may also be employed. These assays are described in Maddox D E et al (1983, J Exp Med 158:1211).

In certain embodiments, the polypeptides and/or antibodies are labeled by joining them, either covalently or noncovalently, with a reporter molecule. A wide variety of reporter molecules are known, including radionuclides, enzymes, fluorescent, chemiluminescent, or chromogenic agents, as well as substrates, cofactors, inhibitors, magnetic particles, and the like. Patents teaching the use of such labels or reporter molecules include: U.S. Pat. Nos. 3,817,837; 3,850,752; 3,939,350; 3,996,345; 4,277,437; 4,275,149, and 4,366,241.

A variety of protocols for measuring TRPM8 receptors, using either polyclonal or monoclonal antibodies specific for the respective protein are known in the art. Examples include enzyme-linked immunosorbent assay (ELISA), radioimmunoassay (RIA) and fluorescent activated cell sorting (FACS).

In order to provide a basis for diagnosis, normal or standard values for TRPM8 receptor expression are established. This is accomplished by combining body fluids or cell extracts taken from normal patient subjects, either animal or human, with antibody to TRPM8 receptors under conditions suitable for complex formation which are well known in the art. The amount of standard complex formation may be quantified by comparing various artificial substrates containing known quantities of TRPM8 receptors with both control and disease samples from biopsied tissues. Then, standard values obtained from normal samples may be compared with values obtained from samples from subjects potentially affected by disease, in particular respiratory disease. Deviation between standard and subject values establishes the presence of disease state.

Polynucleotide sequences encoding TRPM8 receptors may be used for the diagnosis of conditions or diseases with which the expression of TRPM8 receptor is associated. For example, polynucleotide sequences encoding TRPM8 receptor may be used in hybridization or PCR assays of fluids or tissues from biopsies to detect TRPM8 receptor expression. The form of such qualitative or quantitative methods may include Southern or northern analysis, dot blot or other membrane-based technologies; PCR technologies; dip stick, pin, chip and ELISA technologies. All of these techniques are well known in the art and are the basis of many commercially available diagnostic kits.

TRPM8 receptor nucleotide sequences (see sequences provided in Peier, A. M et al., “A TRP channel that senses cold stimuli and menthol,” Cell, 108 (5):705-715 (2002); Brauchi, S. et al., “Clues to understanding cold sensation: thermodynamics and electrophysiological analysis of the cold receptor TRPM8, “Proc. Natl. Acad. Sci. U.S.A., 101 (43):15494-15499 (2004); Bidaux, G. et al., Genbank Accession No. DQ139309; and McKemy, D. D. et al., “Identification of a cold receptor reveals a general role for TRP channels in thermosensation,” Nature, 416 (6876):52-58 (2002); all of which are herein incorporated by reference in their entirety, including any figures, tables, nucleic acid sequences, amino acid sequences, and drawings) provide the basis for assays that detect activation or induction associated with respiratory disease. TRPM8 receptor nucleotide sequence may be labeled by methods known in the art and added to a fluid or tissue sample from a patient under conditions suitable for the formation of hybridization complexes. After an incubation period, the sample is washed with a compatible fluid which optionally contains a dye (or other label requiring a developer). After the compatible fluid is rinsed off, the dye is quantitated and compared with a standard. If the amount of dye in the biopsied or extracted sample is significantly elevated over that of a comparable control sample, the nucleotide sequence has hybridized with nucleotide sequences in the sample, and the presence of elevated levels of TRPM8 receptor nucleotide sequences in the sample indicates the presence of the associated respiratory disease or condition.

Such assays may also be used to evaluate the efficacy of a particular therapeutic treatment regime in animal studies, in clinical trials, or in monitoring the treatment of an individual patient. In order to provide a basis for the diagnosis of disease, a normal or standard profile for TRPM8 receptor expression involved in vagus nerve reflex is established. This is accomplished by combining body fluids or cell extracts taken from normal subjects, either animal or human, with TRPM8 receptor, or a portion thereof, under conditions suitable for hybridization or amplification. Standard hybridization may be quantified by comparing the values obtained for normal subjects with a dilution series of TRPM8 receptor run in the same experiment where a known amount of substantially purified TRPM8 receptor is used. Standard values obtained from normal samples may be compared with values obtained from samples from patients afflicted with TRPM8 receptor-associated diseases, such as respiratory disease. Deviation between standard and subject values is used to establish the presence of disease.

Once disease is established, a therapeutic agent is administered and a treatment profile is generated. Such assays may be repeated on a regular basis to evaluate whether the values in the profile progress toward or return to the normal or standard pattern. Successive treatment profiles may be used to show the efficacy of treatment over a period of several days or several months.

Polymerase Chain Reaction (PCR) as described in U.S. Pat. Nos. 4,683,195 and 4,965,188 provides additional uses for oligonucleotides based upon TRPM8 receptor nucleotide sequence. Such oligomers are generally chemically synthesized, but they may be generated enzymatically or produced from a recombinant source. Oligomers generally comprise two nucleotide sequences, one with sense orientation (5′→3′) and one with antisense (3′→5′), employed under optimized conditions for identification of a specific gene or condition. The same two oligomers, nested sets of oligomers, or even a degenerate pool of oligomers may be employed under less stringent conditions for detection and/or quantitation of closely related DNA or RNA sequences.

Additionally, methods which may be used to quantitate the expression of a particular molecule include radiolabeling (Melby P C et al 1993 J Immunol Methods 159:235-44) or biotinylating (Duplaa C et al 1993 Anal Biochem 229-36) nucleotides, coamplification of a control nucleic acid, and standard curves onto which the experimental results are interpolated. Quantitation of multiple samples may be speeded up by running the assay in an ELISA format where the oligomer of interest is presented in various dilutions and a spectrophotometric or calorimetric response gives rapid quantitation. A definitive diagnosis of this type may allow health professionals to begin aggressive treatment and prevent further worsening of the condition. Similarly, further assays can be used to monitor the progress of a patient during treatment. Furthermore, the nucleotide sequences of TRPM8 receptors may be used in molecular biology techniques that have not yet been developed, provided the new techniques rely on properties of nucleotide sequences that are currently known such as the triplet genetic code, specific base pair interactions, and the like.

### Compositions of the Invention

When reference is made herein to compounds that regulate TRPM8 receptor activity, such reference includes ligands, agonists, antagonists, blockers, and modulators of TRPM8 receptors. In one embodiment, the blocker 2-aminoethoxydiphenylborane is included in therapeutic compositions for use in treating respiratory diseases or conditions. Compounds identified by the methods of the invention can include salts, prodrugs and solvates of the compounds.

The term “salt(s)” as employed herein denotes acidic and/or basic salts formed with inorganic and/or organic acids and bases. Zwitterions (internal or inner salts) are included within the term “salt(s)” as used herein. Also included herein are quaternary ammonium salts such as alkylammonium salts. Pharmaceutically acceptable (i.e., non-toxic, physiologically acceptable) salts are preferred, although other salts are contemplated as within the scope of the invention as they may be useful, for example, in isolation or purification steps employed during preparation. Salts of the compounds of the invention (i.e., 2-aminoethoxydiphenylborane) may be formed, for example, by reacting a compound with an amount of acid or base, such as an equivalent amount, in a medium such as one in which the salt precipitates or in an aqueous medium followed by lyophilization.

Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, 2-hydroxyethanesulfonates, lactates, maleates, methanesulfonates, 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates, undecanoates, and the like.

Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines, N-methyl-D-glucamines, N-methyl-D-glucamides, t-butyl amines, and salts with amino acids such as arginine, lysine and the like. Basic nitrogen-containing groups may be quaternized with agents such as lower alkyl halides (e.g. methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), aralkyl halides (e.g. benzyl and phenethyl bromides), and others.

Prodrugs and solvates of the compounds of the invention are also contemplated herein. The term “prodrug” as employed herein denotes a compound which, upon administration to a subject, undergoes chemical conversion by metabolic or chemical processes to yield a compound of the invention (i.e., 2-aminoethoxydiphenylborane), or a salt and/or solvate thereof.

All stereoisomers of the compounds identified using methods of the invention, including enantiomeric and diastereomeric forms, are contemplated within the scope of this invention. Individual stereoisomers of the compounds of the invention may, for example, be substantially free of other isomers, or may be admixed, for example, as racemates or with all other, or other selected, stereoisomers. The chiral centers of the compounds identified by the present invention can have the S or R configuration as defined by the IUPAC 1974 Recommendations.

According to the invention, compounds that regulate TRPM8 receptor activity, including ligands, agonists, antagonists, blockers, and modulators, are typically employed as part of a pharmaceutical composition including a pharmaceutically-acceptable carrier for treating respiratory and/or non-respiratory diseases. The pharmaceutical compositions comprising at least one compound for regulating TRPM8 receptor activity to treat a respiratory disease or condition, may be formulated, for example, by employing conventional solid or liquid vehicles or diluents, as well as pharmaceutical additives of a type appropriate to the mode of desired administration (for example, excipients, binders, preservatives, stabilizers, flavors, etc.) according to techniques such as those well known in the art of pharmaceutical formulation.

The compounds identified for treating respiratory disease or conditions using the methods of the invention may be administered by any suitable means, for example, orally, such as in the form of tablets, capsules, granules or powders; sublingually; buccally; parenterally, such as by subcutaneous, intravenous, intramuscular, or intrasternal injection or infusion techniques (e.g., as sterile injectable aqueous or non-aqueous solutions or suspensions); nasally, such as by inhalation spray; topically, such as in the form of a cream or ointment; or rectally, such as in the form of suppositories; and in dosage unit formulations containing non-toxic, pharmaceutically-acceptable vehicles or diluents. Compounds that regulate TRPM8 receptor activity may, for example, be administered in a form suitable for immediate release or extended release. Immediate release or extended release may be achieved by the use of suitable pharmaceutical compositions comprising the TRPM8 receptor activity regulating compounds, or, particularly in the case of extended release, by the use of devices such as subcutaneous implants or osmotic pumps. The TRPM8 receptor activity regulating compounds may also be administered in the form of liposomes.

Exemplary compositions for oral administration include suspensions which may contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which may contain, for example, microcrystalline cellulose, dicalcium phosphate, starch, magnesium stearate and/or lactose and/or other excipients, binders, extenders, disintegrants, diluents and lubricants such as those known in the art. The TRPM8 receptor activity regulating compounds of the invention for treating respiratory disease may also be delivered through the oral cavity by sublingual and/or buccal administration. Molded tablets, compressed tablets or freeze-dried tablets are exemplary forms which may be used. Exemplary compositions include those formulating the TRPM8 receptor activity regulating compounds with fast dissolving diluents such as mannitol, lactose, sucrose and/or cyclodextrins. Also included in such formulations may be high molecular weight excipients such as celluloses (avicel) or polyethylene glycols (PEG). Such formulations may also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g., Carbopol 934). Lubricants, glidants, flavors, coloring agents and stabilizers may also be added for ease of fabrication and use.

Exemplary compositions for nasal aerosol or inhalation administration include solutions in saline which may contain, for example, benzyl alcohol or other suitable preservatives, absorption promoters to enhance bioavailability, and/or other solubilizing or dispersing agents such as those known in the art.

Exemplary compositions for parenteral administration include injectable solutions or suspensions which may contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid.

Exemplary compositions for rectal administration include suppositories which may contain, for example, a suitable non-irritating excipient, such as cocoa butter, synthetic glyceride esters or polyethylene glycols, which are solid at ordinary temperatures, but liquefy and/or dissolve in the rectal cavity to release the drug.

Exemplary compositions for topical administration include a topical carrier such as Plastibase (mineral oil gelled with polyethylene).

The effective amount of a compound employed in the present invention may be determined by one of ordinary skill in the art. The effective amount of a compound of the invention preferably refers to that amount of active ingredient, for example an antagonist of TRPM8 receptors, which ameliorates the symptoms or conditions associated with a respiratory disease. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage form employed, sensitivity of the patient, and the route of administration.

For example, where a TRPM8 receptor blocker, such as 2-aminoethoxydiphenylborane or compounds therefrom, is to be administered to a patient to treat a respiratory disease or condition, exemplary dosage amounts for an adult human include from about 0.1 to 1,000 mg/kg of body weight of active compound per day, which may be administered in a single dose or in the form of individual divided doses, such as from 1 to 4 times per day. It will be understood that the specific dose level and frequency of dosage for any particular compound may be varied and will depend upon a variety of factors including the activity of the specific compound employed, the metabolic stability and length of action of that compound, the species, age, body weight, general health, sex and diet of the patient, the mode and time of administration, rate of excretion, drug combination, and severity of the particular condition.

### Methods for Treating Respiratory Diseases or Conditions

According to the subject invention, TRPM8 receptors are molecules involved in cold-induced vagus nerve reflex. Without being bound by any one theory, TRPM8 receptors appear to be expressed on the vagus nerves of the bronchopulmonary system.

In one embodiment, vectors expressing TRPM8 receptors may be administered to a patient to increase the level of TRPM8 receptors in conditions characterized by defective functioning of the vagus nerve reflex and/or low levels of TRPM8 expression.

In another embodiment of the invention, gene therapy that manipulates TRPM8 receptor activity can be administered to a patient diagnosed with a respiratory disease or condition. For example, gene transfer of antisense RNA sequence using viral vector systems to silence TRPM8 receptor activity can be used in accordance with the subject invention to treat a respiratory disease or condition. Exemplary methods for gene therapy based on the manipulation of TRPM8 receptor activity include those disclosed by Yoshimura, K. et al., “Expression of the human cystic fibrosis transmembrane conductance regulator gene in the mouse lung after in vivo intratracheal plasmid-mediated gene transfer,” Nuc Acids Res., 20 (12):3233-3240 (1992) and Mangeot, P. E. et al., “A universal transgene silencing method based on RNA interference,” Nuc Acids Res., 32 (12):e102 (2004). Sense or antisense oligomers or larger fragments can be designed from various locations along the coding or control regions of sequence encoding TRPM8 receptors.

A variety of expression vector/host systems may be utilized to contain and express sequences encoding TRPM8 receptors. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems. Methods that are well known to those skilled in the art can be used to construct recombinant vectors that will express antisense molecules complementary to the polynucleotides of the gene encoding the TRPM8 receptors. These techniques are described both in Yoshimura et al. (supra) and Mangeot et al. (supra).

Antisense molecules and ribozymes of the invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding TRPM8 receptors. Such DNA sequences may be incorporated into a wide variety of vectors with suitable RNA polymerase promoters. Alternatively, these cDNA constructs that synthesize antisense RNA constitutively or inducibly can be introduced into cell lines, cells, or tissues.

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection and by liposome injections may be achieved using methods which are well known in the art.

In another embodiment of the invention, an effective amount of a compound that binds to TRPM8 receptors to modulate TRPM8 activity is administered to a patient diagnosed with a respiratory disease or condition. In related embodiments, antagonists or inhibitors of TRPM8 receptors are administered to a patient to suppress expression of TRPM8 receptors for the treatment of respiratory diseases or condition including, but not limited to, cold-induced asthma or asthma-exacerbation. Following are examples, which illustrate procedures for practicing the invention. These examples should not be construed as limiting. All percentages are by weight and all solvent mixture proportions are by volume unless otherwise noted.

### EXAMPLE I

### Characteristic of TRPM8 Receptor Activity in Vagus Nervous System

### 1. Tissue Collections

Tissues from any patient (i.e., primates, humans, domesticated animals) are used to study TRPM8 receptor activity in vagus nerves to assess vagus nerve reflex of the bronchopulmonary system. Preferably, human tissues are utilized. The skilled artisan would readily grasp those tissues useful in studying TRPM8 receptor activity, which can include bronchopulmonary tissues and the ganglions of the vagus nerves. The ganglions are used to determine whether the cell body of the vagus afferent fibers expresses TRPM8 receptors. The bronchopulmonary tissues are used to determine whether the afferent nerve endings that innervate bronchopulmonary tissues express TRPM8 receptors.

Human bronchopulmanary tissues can be obtained from any known resource including, for example, the Molecular Tissue Bank (MTB) at the University of Florida and the Pulmonary Clinic at the Shands Hospital of the University of Florida. These two sources currently have “normal” bronchopulmonary tissues and the tissues of different bronchopulmonary disorders including asthma. According to certain examples provided below, the ganglions of the vagus nerve are obtained from the Human Brain Tissue Bank at the University of Florida and the Pathserve & Autopsy and Human Tissue Bank (a tissue bank located in San Francisco). All tissues are snap-frozen, which are suitable for the exemplary studies described herein.

### 2. Development of Antibodies Against TRPM8 Receptors

The skilled artisan would readily recognize the many available assays suitable for identifying TRPM8 receptors. According to certain embodiments of the invention, either immunochemistry and/or western blot, using TRPM8 antibody, can be used to study TRPM8 receptors on patient tissues. Using antibodies against TRPM8 receptors, the expression of TRPM8 receptors on vagus ganglion neurons and dorsal root ganglion neurons can be demonstrated. In one embodiment, the antibody used is produced from an animal (i.e., rabbit) and is against the N-terminals of TRPM8 receptors. For example, the antibody to a human TRPM8 receptor antibody can be produced using different amino acid sequences in the TRPM8 receptors.

To verify antibody recognition of TRPM8 receptors, on embodiment of the invention expresses TRPM8 receptors on commercially available heterologous expression systems (i.e., for human TRPM8 receptors, HE 93 cell line). Human TRPM8 cDNAs are currently available from several sources (see, for example, those disclosed in Brauchi, S. et al., “Clues to understanding cold sensation: thermodynamics and electrophysiological analysis of the cold receptor TRPM8,” Proc. Natl. Acad. Sci. U.S.A., 101 (43):15494-15499 (2004); and Bidaux, G. et al., Genbank Accession No. DQ139309). Following the expression of human TRPM8 receptors on HEK239 cells, immunostaining and western blot are performed to see whether the antibody recognizes the TRPM8 receptors.

3. TRPM8 Expression on Ganglion Neurons of Vagus Nerve and on the Nerve Endings that Innervate Bronchopulmonary Tissues

In one embodiment, retrograde labeling of vagus ganglions or bronchopulmonary tissues is performed on a murine or rat model. This is performed by instillation of a small amount of nerve tracing dye (such as DiI, 1,1′-dioctadecyl-3,3,3′,3′-tetramethylindocarbocyanine percholate, Molecular Probes, Eugene Oreg.) into the lower part of the trachea. Instillation of the nerve tracing dye in the lower part of mice trachea was performed and, after 7 days, the mice (or rats) were sacrificed and the vagus ganglions were harvested, dissociated, and plated in dishes. Calcium-imaging approach was then used to identify bronchopulmonary vagus afferent neuron response to cold and menthol. With Ca²⁺-imaging, relative fluorescent intensity ΔF/F₀ was used and a ΔF/F₀ value of >0.1 was considered to have response. All the data were represented as mean ±SEM. Paired-t tests were used for statistical comparison, and significance was considered at the p<0.05.

To perform calcium imaging, VGNs were incubated with 2 μM Fluo3-AM in 20% pluronic acid (Molecular Probes, Eugene, Oreg.) for 30 min at 37° C. to load Ca²⁺ indicator. The cells were then perfused with normal bath solution flowing at 1 ml/min in a 0.5 ml chamber. The normal bath solution contained (in mM): 150 NaCl, 5 KCl, 2 MgCl₂, 2 CaCl₂, 10 glucose, 10 HEPES, pH 7.2, osmolarity adjusted to 320 mOsm with sucrose. Fluo-3 fluorescence in the cells was detected using a peltier-cooled charge-coupled device (CCD) camera (PentaMAX-III System, Roper Scientific, Trenton, N.J.) under a fluorescent microscope (10× objective). Excitation was at 450 nm and emission at 550 nm, achieved by a fluorescence filter sets. Images were taken at one frame per second, and digitized using MetaFluor software (Universal Imaging Corporation). Unless otherwise indicated, all experiments were carried out at an ambient temperature of 26° C. Effects of cold on intracellular Ca²⁺ levels were tested by application of a cold bath solution, which yield temperature drop from 26° C. to 19° C. within 1 min in the recording chamber. Effects of menthol were tested by application of 100 μM menthol for 20 s. Cold and menthol solutions were delivered through a glass tube; the tube had internal diameter of 0.5 mm and was positioned 0.5 mm away from the recorded cells.

Tissue sections (14 μm in thickness) are cut on a cryostat and thaw-mounted onto slides. Dry slide-mounted sections are encircled with hydrophobic resin (PAP Pen; The Binding Site) and first incubated in 1:30 normal goat serum (GS) in PBS with 0.4% Triton X-100 (T) for 1 hour to block non-specific binding. The sections are then incubated overnight in a solution of rabbit anti-TRPM8 antibody diluted 1:500 in 1% GS-PBS-T. The sections are rinsed repeatedly in 1% GS-PBS-T. They are incubated for 3 h in a 1:300 solution of biotin conjugated goat-anti-rabbit IgG (Jackson ImmunoResearch). The cells are further rinsed with repeated applications of 1% GS-PBS-T. They are then incubated for 40 min in a 1:100 solution of streptavidin-Alexa Fluor 488 (Molecular probes). The sections are rinsed with 1% GS-PBS-T and are then coverslipped with a glycerol-based photobleach protective medium.

In one embodiment, Adult Sprague-Dawley rats (200-250 g, n=6) were perfusion fixed and the inferior parts of vagus sensory ganglia (nodose) were dissected out. The ganglia were cut with a cryostat into transverse sections at thickness of 16 μm. Sections were incubated in a rabbit anti-TRPM8 (1:300) overnight at 4° C. and then 3 hrs with a secondary antibody (goat-anti-rabbit, conjugated with Alexa 594). The TRPM8 antibody was generated in rabbits using a sequence of N-terminus, EGARLSMRSRRNG, of rat TRPM8 receptors. Double immunostaining of TRPM8 and P2X₃ receptors were performed by first incubating ganglia sections with a guinea pig anti-P2X3 antibody (1:3000) overnight at 4° C., followed by a 3 hrs of incubation with a secondary antibody (goat-anti-guinea, conjugated with Alexa 488). Immunostaining for TRPM8 receptors were subsequently performed as described above. Images were acquired using a fluorescence microscope. To determine IR positive neurons in each dorsal root ganglion, a threshold is set at 2.5 times of averaged cytoplasmic density level. All neurons sectioned through their nucleus for which mean optical density exceeded the threshold are counted as positive.

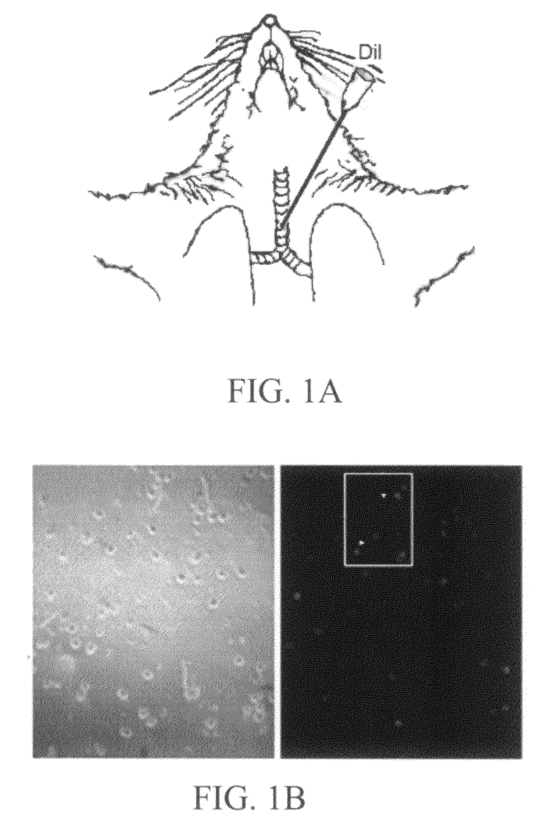

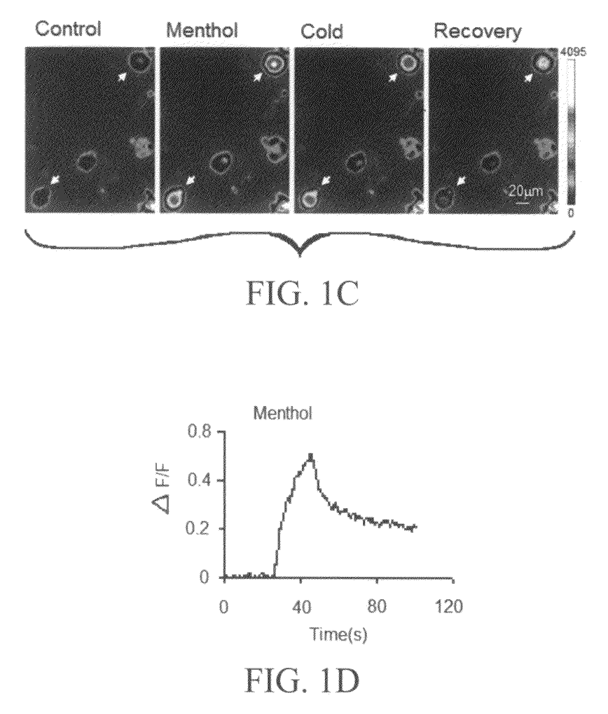

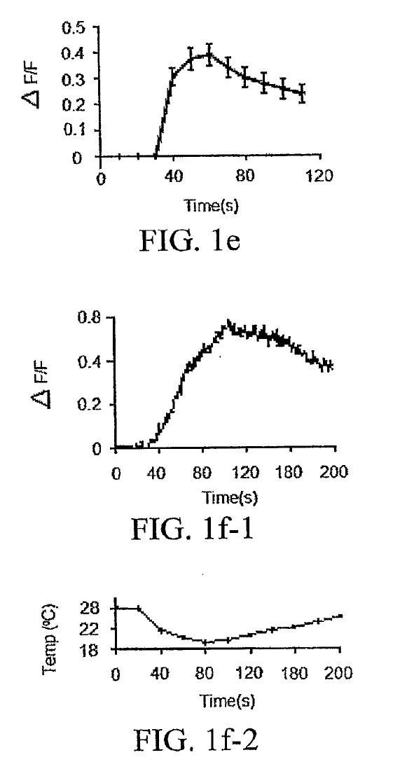

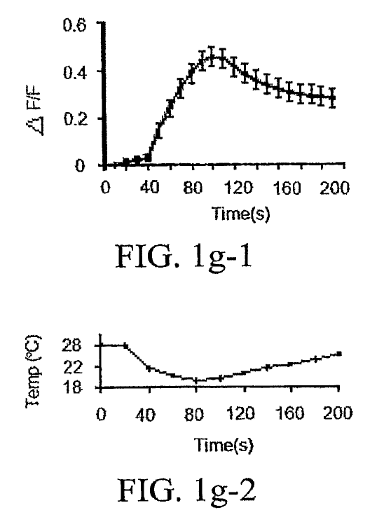

In a related embodiment, Adult Sprague-Dawley rats (200 to 300 g, n=48) were used according to the Institutional Animal Care and Use Committee guideline of the University of Florida. The rats were continuously anesthetized with isoflurane using an anaesthetizing machine. To retrograde label the vagus ganglion neurons that innervate low airway tissues, a 20 μl-DiI was instilled into the caudal region of the trachea in living adult rats (see FIG. 1a, which illustrates the instillation of DiI into the lower segment of rat trachea to retrograde label airway VGNs). The animals were positioned supine during dye instillation and kept the same position for 30 min before recovery from anesthesia. Seven days after dye instillation, the inferior parts of vagus nerve ganglia were removed from the rats. The ganglions were incubated for 45 min at 37° C. in S-MEM medium (Gibco, Grand Island, N.Y.) with 0.2% collagenase and 1% dispase (Sigma) and then triturated to dissociate neurons. The neurons were plated on glass coverslips previously covered with PDL and used within 4 hours after plating.

Of 1638 acutely dissociated VGNs, 170 of them showed a high intensity of DiI labeling (see FIG. 1b, which displays micrographs of a field of acutely dissociated VGNs under bright light (left panel) and fluorescent light (right panel)). The effects of TRPM8 receptor ligand menthol on the airway VGNs were analyzed using a Ca²⁺ imaging technique.

As shown in FIG. 1c-e, application of menthol (100 μM, 20 s) resulted in increases of intracellular Ca²⁺ in 7% of VGNs (116/1638 cells), including both DiI-labeled and non-labeled neurons. Specifically, FIG. 1c provides images of menthol versus cold-induced increases of [Ca²⁺]ᵢₙ in DiI-labeled VGNs during different times. FIG. 1c provides an image of [Ca²⁺]ᵢₙ levels taken before either menthol- or cold-application (control), an image of [Ca²⁺]ᵢₙ levels taken following a 20-second application of 100 μM menthol, an image of [Ca²⁺]ᵢₙ levels taken following a 7° C. temperature drop, and an image of [Ca²⁺]ᵢₙ levels taken during recovery in normal bath solution. FIGS. 1d and 1e are graphical illustrations of time course menthol induced responses in a DiI-labeled neuron versus 85 neurons (pooled results). Of the 170 DiI-labeled cells, 28 of them (17%) showed response to menthol (FIG. 1c).

According to the subject invention, additional study was performed to assess whether cold stimuli might produce a similar response to menthol on VGNs. Since there was little activation of TRPM8 receptors at temperatures of 24-28° C. or above, a basal temperature of 26° C. was used in the study. As shown in FIGS. 1c, 1f, and 1g, a 7° C. of temperature drop, from 26° C. to 19° C., resulted in increases of intracellular Ca²⁺ in 6.5% of VGNs (106/1638 cells), including both DiI-labeled and non-labeled neurons. FIGS. 1f and 1g are graphical illustrations of time course cold induced responses in DiI-labeled neurons versus 85 neurons (pooled results), where the intensity changes of the Ca²⁺ indicator Fluo-3 (denoted as ΔF/F₀) were shown on the top and the temperature ramps on the bottom. All these cold-responsive cells were menthol-responsive neurons. Among the 170 DiI-labeled cells, 27 of them (16%) were both menthol- and cold-responsive neurons. These results indicate that a subpopulation of VGNs innervating respiratory airways responds to both cold and the TRPM8 receptor agonist menthol.

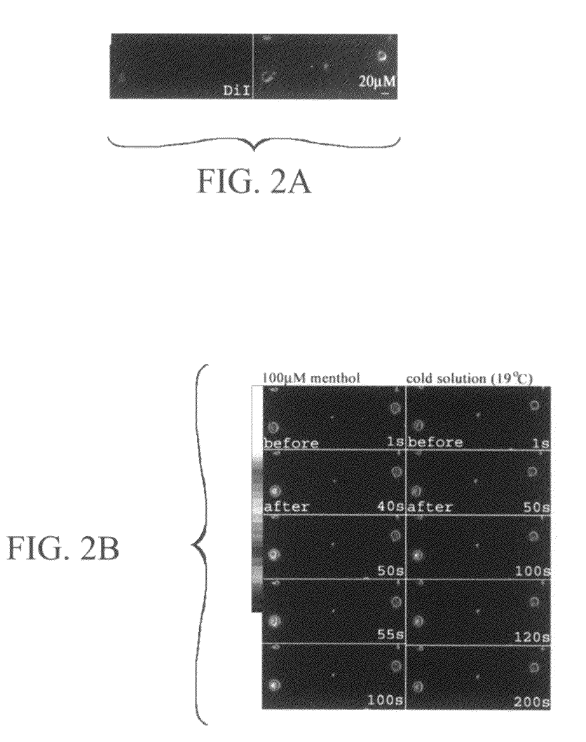

Such results indicate that cold temperature and cooling compounds stimulate bronchopulmonary afferent fibers of rat vagus nerve and that the effects are mediated by TRPM8 receptors, see also FIGS. 2a and 2b. FIG. 2a displays a micrograph (with a left and right panel), where the left panel shows a retrogradely labeled bronchopulmonary vagus afferent neuron. The right panel is a bright filled image of the same cells. FIG. 2b shows responses of the bronchopulmonary vagus ganglion neuron to menthol and cold. Using the plated vagus ganglions, menthol (100 μM) was applied at 20 second time point and terminated at 40 second time point; cold bath solution was applied at 20 second time point and ended at 30 second time point with the temperature drop from 26° C. to 19° C. In the left panel, a series of images (from top to bottom) show that the retrogradely labeled neuron “changed color” following the application of 100 μM menthol. In the same field, another cell (non-labeled neuron) did not have a response. In the right panel of FIG. 2b, a series of images (from top to bottom) show that the retrogradely labeled neuron also “changed color” following the application of cold bath solution. The non-labeled cell did not respond to cold.

In a related embodiment, dorsal root ganglion neurons were obtained using procedures described above. DiI-labeled VGNs were identified under the fluorescence microscope (excitation, 550 nm; emission, 650 nm). Seven days after this procedure, mice were perfusion-fixed and vagus ganglions were harvested for immunostaining using TRPM8 antibody. Where TRPM8-immunoreactivity was found on DiI labeled neurons, it indicated that TRPM8 receptors were expressed on vagus afferent fibers innervating bronchopulmonary tissue in mice. Immunostaining results were used to identify the percentage of DiI labeled neurons that are TRPM8 immunoreactive neurons.

### 4. TRPM8 Protein Expression on VGNs

According to the subject invention, studies are performed to assess whether TRPM8 proteins were expressed on VGNs by immunostaining sections of VGN ganglion using a TRPM8 antibody. In one study, results regarding TRPM8 receptor expression on rat bronchopulmonary afferent fiber are illustrated in FIGS. 3a and 3b. FIGS. 3a and 3b demonstrate TRPM8 receptor expression on the ganglion neurons of vagus nerve using an antibody against TRPM8 receptors. Specifically, FIG. 3a is an image showing TRPM8 immunoreactivity (TRPM8-ir) in an inferior part of vagus ganglion section of an adult rat (scale bar: 30 μm). FIG. 3b is a histogram illustrating the size distribution of TRPM8-ir vagus ganglion neurons. The total number of TRPM8-ir positive neurons are 184, pooled from 6 vagus ganglia of 3 rats.

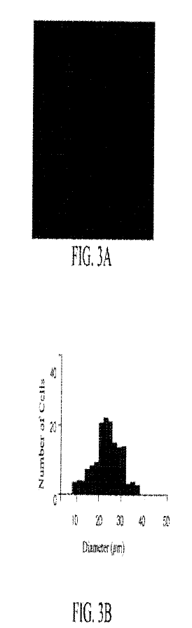

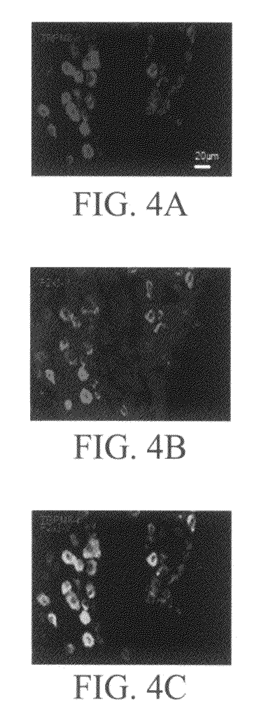

It is noted that most TRPM8-ir positive neurons have a diameter smaller than 30 μm In another study, TRPM8 immunoreactivity (-ir) was found to be present in 7.2% of VGNs (75/1041 cells, FIG. 4a, which is an image of TRPM8 immunoreactivity in a portion of an inferior part of a vagus ganglion section). As illustrated in FIGS. 4a through 4c, the cell sizes were 24±0.7 μm, ranging from 12 to 36 μm.

Many vagal afferent nerves innervating respiratory airways express P2X3 receptors and these nerves were suggested to be involved in vagal nerve reflex (Brouns et al, 2003). To determine whether TRPM8 receptors may be located on P2×3-expressing vagal neurons, double immunostaining was used with both the TRPM8 antibody and a P2X3 receptor antibody. About 40% of VGNs (415/1041 cells, FIG. 4b) were found to be P2×3-ir positive. The TRPM8-ir and P2×3-ir double positive neurons accounted for majority (68%) of TRPM8-ir positive neurons (see FIG. 4c, which is an image of P2X3 immunoreactivity in the same vagus ganglion section as shown in FIG. 4a).

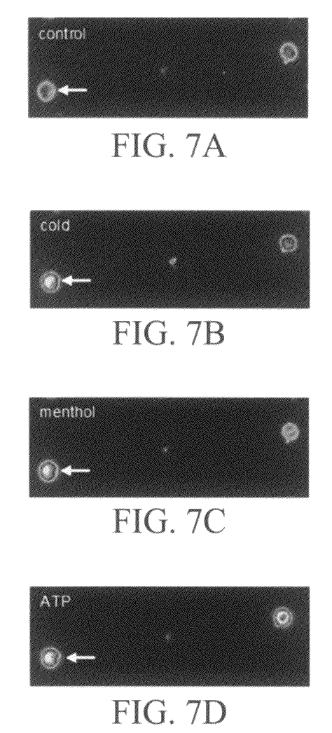

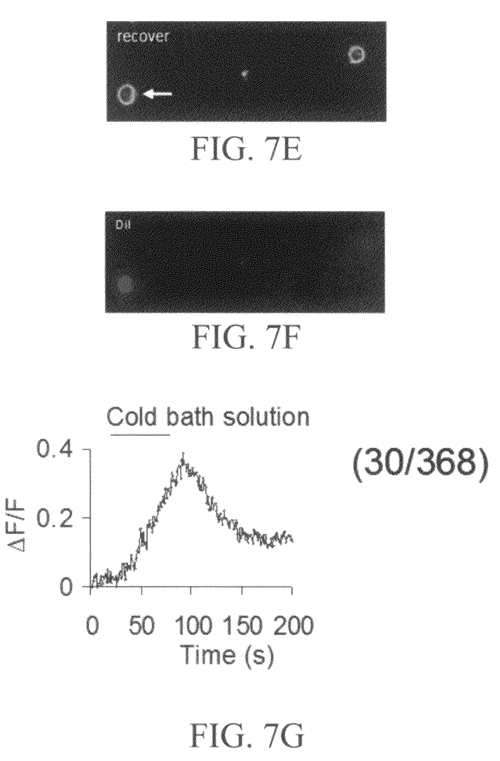

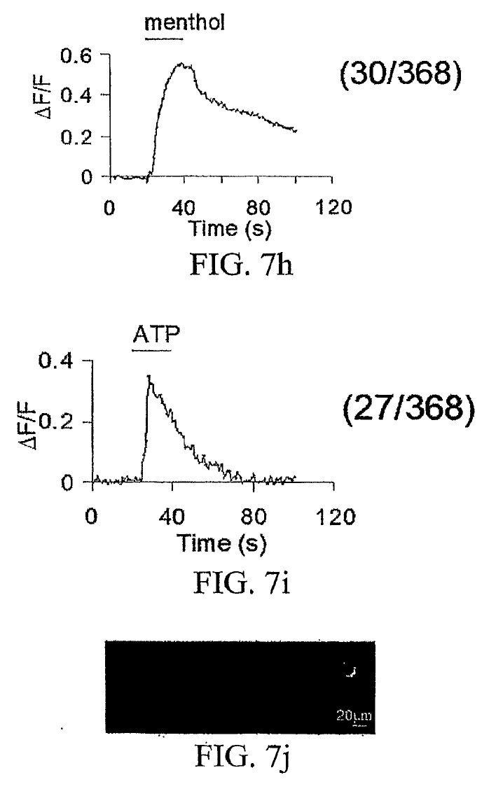

When menthol and ATP responses were tested using Ca²⁺ imaging technique, it was found that almost all menthol-responsive VGNs were ATP-sensitive (FIGS. 7a through 7j). These results indicate that TRMP8 receptors are expressed predominantly on a subpopulation of VGNs that are ATP-sensitive. The results also suggest that endogenously released ATP (presumably due to bronchopulmonary tissue stretch, Brouns et al., 2003) and cold stimuli have a synergistic effect to provoke bronchopulmonary autonomic reflex.

### 5. Electrophysiological Properties of Cold- and Menthol-Induced Responses

In certain embodiments of the invention, whole-cell recordings were performed on VGNs. For most voltage-clamp recordings, electrode internal solution contained (in mM): 110 Cs₂SO₄, 2 MgCl₂, 0.5 CaCl₂, 5 TEA-Cl, 5 EGTA, 5 HEPES, pH 7.3. When both voltage-clamp and current-clamp recordings were applied to the same cells, electrode intracellular solution contained (in mM) 135 K-gluconate, 2 MgCl₂, 0.5 CaCl₂, 5 EGTA, 10 HEPES, 2 Na₂ATP, 0.5 NaGTP, pH 7.3. Recording electrode resistance was ˜5 MΩ. Unless otherwise indicated, voltage-clamp recordings were performed on cells held at −70 mV. Signals were amplified with MultiClamp 700A (Axon Instruments, Union City, Calif.), filtered at 2 kHz and sampled at 5 kHz. Cold bath solution and menthol were applied to neurons in the same manner as that in Ca⁺-imaging experiments. A voltage ramp from −90 mV to 70 mV was used to obtain I-V relationship of menthol-evoked currents. The voltage ramp was applied during the steady states of menthol-evoked currents. I-V relationship was constructed after a subtraction of a control ramp test. During the voltage-ramp test, lidocaine (1 mM) was used to block both TTX-sensitive and TTX-resistant Na⁺ channels and Ca²⁺ channels.

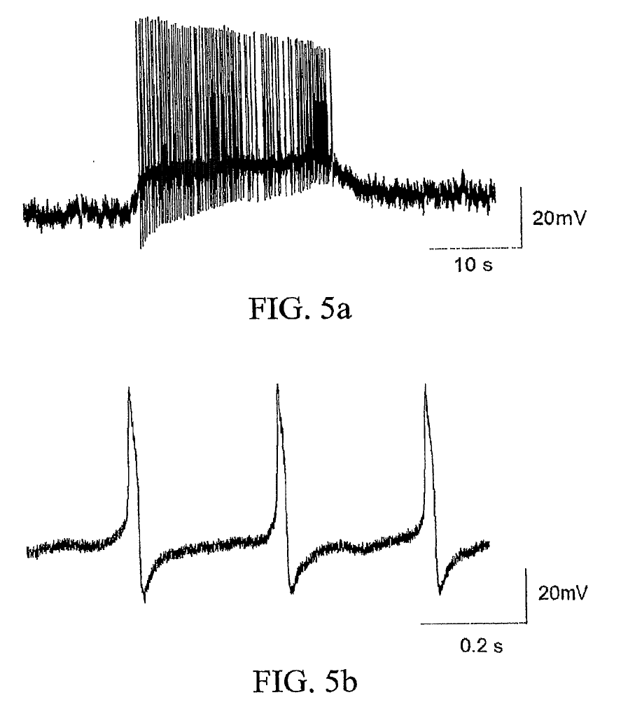

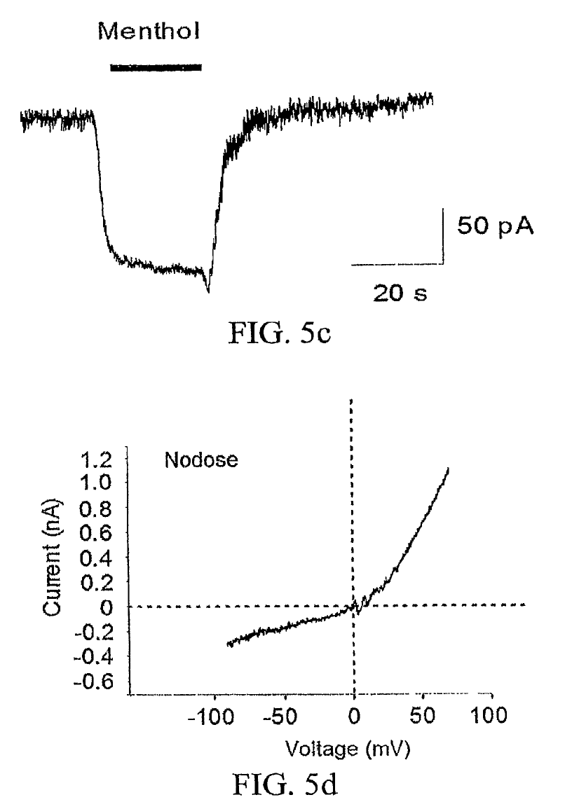

FIGS. 5a-5d illustrate the results of electrophysiological screening methods that were conducted to confirm TRPM8 receptors expression on ganglion neurons of the vagus nerve and that TRPM8 activation excite these neurons. The recordings were made from menthol-sensitive neurons pre-identified using Ca²⁺-imaging technique as shown in FIGS. 2a and 2b.

FIG. 5a are traces that show action potentials elicited by 100 μM menthol on a vagus ganglion neuron. The recording was performed under current-clamp configuration with a K⁺-containing electrode. FIG. 5b are the traces in FIG. 5a on an expanded scale. FIG. 5b is a sample trace showing a whole-cell inward current evoked from a vagus ganglion neuron following a 20-second application of menthol (100 μM). Holding potential was −70 mV. Similar results were obtained from 6 other cells. FIG. 5d illustrates current-voltage relationship. Specifically demonstrated in FIG. 5d is the outward rectification of menthol-evoked currents from a vagus ganglion neuron. The cell was held at −70 and 100 M menthol was applied for 20 seconds. At the 10^{th} second of menthol application, a voltage ramp was applied, from −90 to +140 mV in 500 milliseconds. Similar results were obtained from 2 other cells. The results illustrated in FIGS. 5a-5d provide electrophysiological evidence for the expression of TRPM8 receptors on the vagus ganglion neurons and indicate the excitatory function of these receptors on these cells.

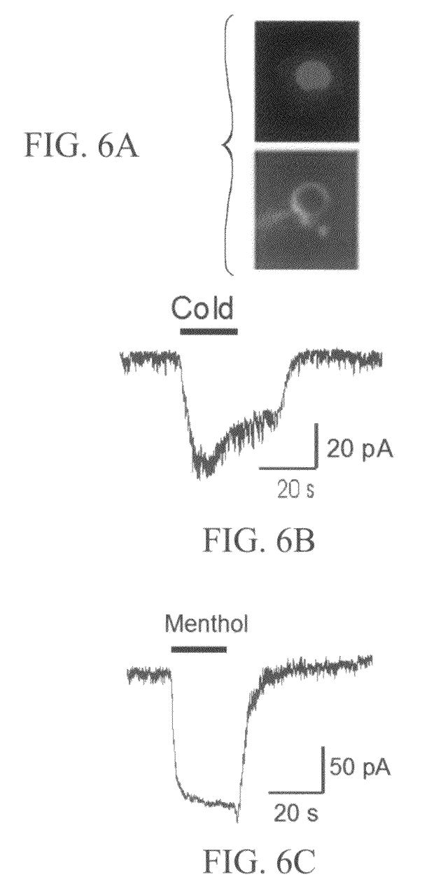

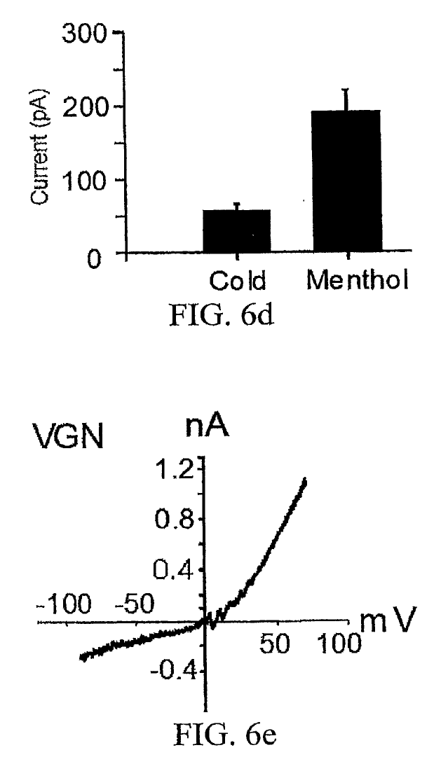

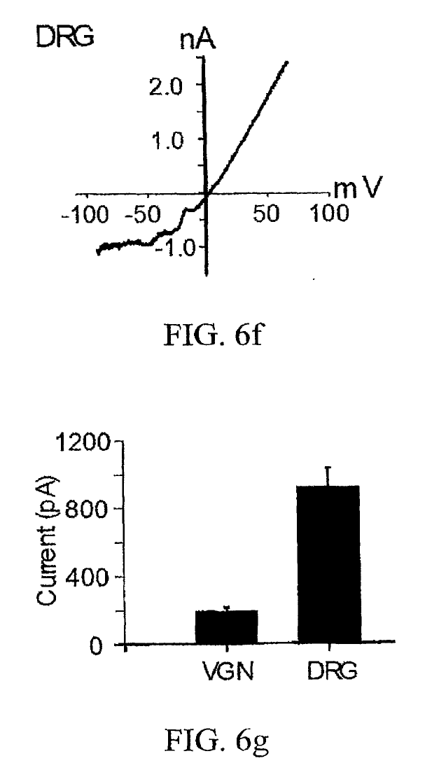

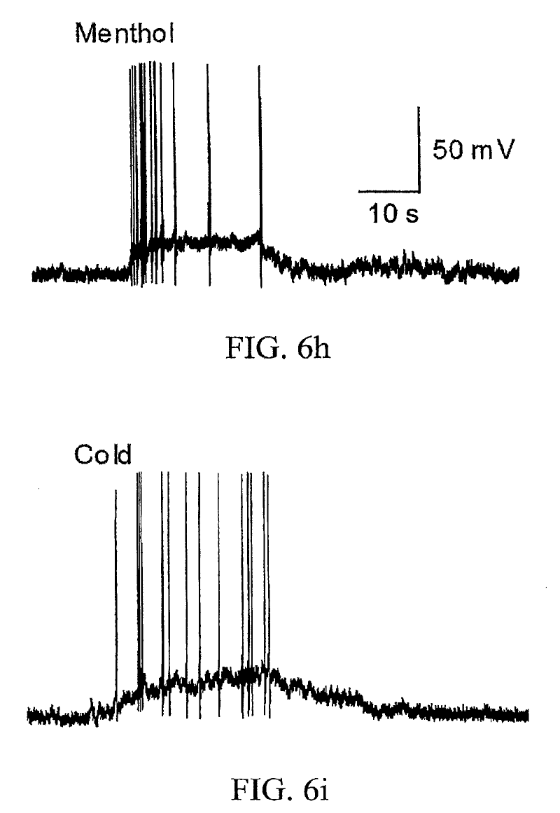

In a related embodiment, another study was performed to confirm that the VGNs responses to cold and menthol were mediated by TRPM8 receptors. In this study, electrophysiological properties of cold- and menthol-induced responses were characterized. FIG. 6a shows a DiI labeled VGN that was included in patch-clamp recording. All neurons used for the electrophysiological study were pre-identified to be menthol-responsive VGNs using the Ca²⁺ imaging technique described earlier. Under voltage-clamp configuration with cells held at −70 mV, a temperature drop from 26° C. to 21° C. within 20 sec evoked inward currents (FIGS. 6b and 6d, 57±9 pA, n=11). Menthol (100 μM, 20 s) was tested subsequently in 7 cells and all of them showed inward currents (FIGS. 6c and 6d, 191±29 pA, n=7). The relationship between menthol-evoked currents and holding potentials showed strong outward rectification with a reversal potential near 0 mV (−2±0.3 mV, n=4, FIG. 6e). These properties are consistent with the electrophysiological characteristics of the cloned TRPM8 that were expressed in heterologous expression system (McKemy et al., 2002; Peier at al., 2002) as well as the characteristics of cold-sensing somatic sensory neurons acutely dissociated from rat dorsal root ganglions (DRGs) (FIG. 6f). These results indicate that cold and menthol responses in respiratory airway VGNs were mediated by TRPM8 receptors.

TRPM8 currents in VGN neurons were found to be substantially smaller than those in DRG neurons (FIG. 6g). To assess whether TRPM8 activation is sufficient to excite VGNs, recordings were made of VGNs under current-clamp configuration and the effect of menthol and cold were tested. Menthol (100 μM, 20 s) depolarized VGN neurons from the resting membrane potential of 60 mV (60±0.6 mV) to 38 mV (38±1.5 mV) and caused action potential firing (n=4, FIG. 6h). Similar to the response to menthol, a temperature drop from 26 to 21° C. within 20 s depolarized VGN neurons from the resting membrane potential of 61 mV (61±0.4 mV) to 36 mV (36±2.1 mV), which was followed by action potential firing (n=4, FIG. 6i). These results indicate that a 5° C. of temperature drop to 21° C. is sufficient to excite respiratory airway VGNs. It has been shown that air temperature in human tracheas can reach near 20° C. or lower when breathing cold air in situations such as winter sports. Thus, the cooling temperatures used demonstrated in the subject study are relevant to cold-induced asthma attack.

### 6. TRPM8 Receptor Involvement in Cold-Induced Asthmatic Reaction

According to the subject invention, inhalation of a high concentration of menthol (1 mM), an agonist of TRPM8 receptors, results in a breathing difficulty in rats (n=3) although the respiratory reaction is not obvious with a low concentration of menthol (1 μM).

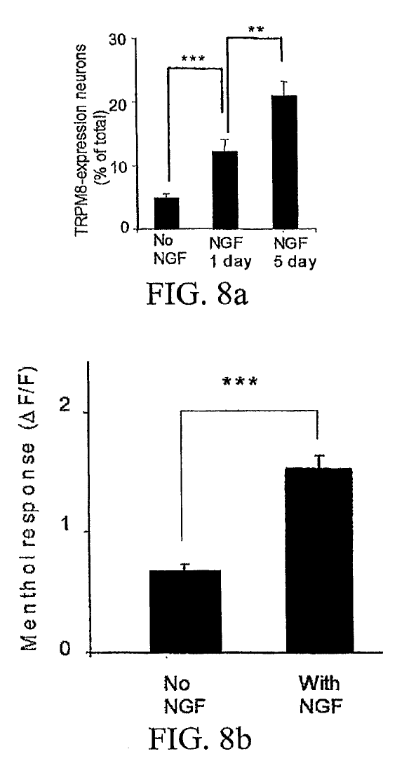

Inflammation in lung tissues often can result in the increases the levels of nerve growth factor (NGF). According to the subject invention, the expression of TRPM8 receptors can be up-regulated by NGF. Upregulation of TRPM8 receptors on these nerves can sensitize their responses to cold stimulation. FIGS. 8a and 8b illustrate the numbers of TRPM8 expression neurons and the responsiveness of these neurons to the TRPM8 ligand menthol. Specifically, FIG. 8a is a graphical illustration of an increase in number of TRPM8-expressing neurons in cell culture when in the presence of nerve growth factor (NGF). FIG. 1b is a graphical illustration of the response of TRPM8-expressing neurons to the TRPM8 receptor agonist menthol when cultured in the presence or absence of NGF. The one day cultures were used in the absence of NGF.

### 7. TRPM8 Protein Presence in Bronchopulmonary Tissues

Vagus afferent nerve endings that innervate bronchopulmonary tissues may express TRPM8 proteins at different levels under normal and disease conditions. Using the methods described in Example 1, TRPM8 expression in normal bronchopulmonary tissues can be compared against bronchopulmonary tissues from asthma subject without smoking history and bronchopulmonary tissues from asthma with smoking history. This comparison study provides information regarding whether TRPM8 expression is altered under disease conditions and if so, whether the change may be associated with smoking. Western Blot with the use of TRPM8 antibody can be applied to the different sets of tissues.

### EXAMPLE 2

### Functional Screening of TRPM8 Receptor Activity

In a patient, the vagus nerve afferent fibers that innervate bronchopulmonary tissues are retrogradely labeled using DiI, as described above in Example 1. Seven days following this nerve tracing procedure, the vagus nerve ganglions are harvested and the ganglion neurons are acutely dissociated. The dissociated neurons are plated in the dishes and used for high-throughput calcium-imaging and patch-clamp recording experiments within 6 hours. Dissociate neuron preparation may be utilized to ensure healthy neurons that are suitable for in vitro functional study.

To determine cold response by calcium-imaging technique, neurons will be first loaded with the Ca²⁺ indicator Fluo-3. This is done by incubating neurons with 5 μM Fluo-3-AM in 20% pluronic acid (Molecular Probes, Eugene, Oreg.) for 30 min at 35° C. After dye loading, the cells are perfused with normal bath solution in a 0.5 ml chamber on a microscope stage (Olympus IX70). The normal bath solution contained (in mM): 150 NaCl, 5 KCl, 2 MgCl₂, 0.5 CaCl₂, 10 glucose, 10 HEPES, pH 7.2, osmolarity adjusted to 320 mOsm with sucrose, temperature at 35° C. Fluo-3 fluorescence in the cells are detected with a peltier-cooled charge-coupled device (CCD) camera (PentaMAX-III System, Roper Scientific, Trenton, N.J.) under a 20× objective. Excitation is at 450 nm and emission at 550 nm, achieved by a fluorescence filter set. Images are acquired at one frame per second during the application of cold solution. Cold solution will be application to produce a temperature drop from 35° C. to 18° C. within 1 min. Increases of fluorescence intensity in dye-labeled neurons will indicate the increases of intracellular Ca²⁺ in response to cold stimulation. For these cold responsive neurons, their response to menthol will also be assessed, where menthol is an agonist for the TRPM8 receptor. Menthol (100 μM) will be applied for 20 seconds. It is predicted that the cold-responsive cells will also respond to menthol by an increase of intracellular Ca²⁺.

To confirm that cold and menthol responses of these neurons are indeed mediated by the activation of TRPM8 receptors, patch-clamp recordings are performed on vagus ganglion neurons. Neurons will be under voltage-clamp configuration held at −70 mV with electrodes containing CsSO₄ internal solution. Cold and menthol responses will be initiated by the application of cold or menthol in the same ways as those in Ca²⁺ imaging experiments. Whether cold and menthol produce inward currents with reversal potentials near 0 mV (use voltage ramp test) can then be assessed. This indicates that these neurons respond to cold and menthol through the activation of TRPM receptors.

### EXAMPLE 3

### TRPM8 Receptor Activity in Relation to Substance P

The nociceptive mediator substance P (SP) that is present in somatic sensory afferent fibers has been found in the vagus afferent fibers. SP released from the vagus afferent fibers are involved in inflammatory reaction in bronchopulmonary tissues under a number of respiratory pathological conditions including asthma. Because SP plays a pathological role in bronchopulmonary inflammation and asthma, it is beneficial to detect SP release from autonomic sensory neurons (as opposed to previously disclosed somatosensory neurons). Further, by analyzing of the relationship between TRPM8 receptor activity and SP release, the subject invention provides important information regarding the pathology of asthma and other respiratory diseases for use in screening methods and/or compositions useful in the diagnosis or treatment of respiratory diseases.

To determine whether TRPM8-expressing vagus nerves are neuropeptidergic afferent fibers that contain substance P, the tissues of Example 1 can be used. Following the overnight incubation with anti-TRPM8 antibody (as described above in Example 1), tissue sections are incubated over sequential evenings in solutions of polyclonal anti-SP (1:3000; Peninsula Labs). The primary antisera are detected each following day by incubation with the appropriate species-specific secondary antisera (raised in goat) conjugated with either AlexaFluor 594 (red) and AlexaFluor 488 (green). If TRPM8-expressing vagus nerves contain substance P, action of TRPM8 receptors can cause SP release to produce neurogenic inflammation.

The immunostaining of tissues described above enables evaluation of SP depletion (i.e., substance release) following chemical stimulation. The same approach can be used to show that activation of TRPM8 receptors can release SP from vagus ganglion neurons. In such experiments, vagus ganglion neurons are plated in culture dish (35 mm dishes) for two weeks. Cells are exposed to either cold (17° C.) for 30 minutes or the TRPM8 agonist menthol (100 μM) for 10 minutes. Following these treatments, immunostaining of SP can be performed using an SP antibody. The immunostaining results of treated cells can then be compared against those untreated cells to determine whether SP becomes depleted (i.e., released) following the treatments.

To quantitatively measure SP release following stimulation by cold or menthol, the medium in the culture dish will be collected to detect the concentration of SP using enzyme-linked immunoabsorbent assay (ELISA). The basal SP levels in dishes without treatment will be compared against SP levels following treatments.

In a specific embodiment, a study can be performed to determine whether TRPM8-expressing vagus afferent nerve fibers are SP-containing afferent nerves. In this set of experiments (n=12), immunostaining for SP will be performed following the immunostaining of TRPM8 as described in Example 1 to determine if SP-containing neurons express TRPM8 receptors.

In a related embodiment, vagus ganglion neurons are dissociated and plated in 35-mm dishes. Cells are then be exposed to either a cold bath solution (19° C.) for 10 min (n=12), or the TRPM8 agonist menthol (100 μM) for 10 min (n=12). Following these treatments, media in the dishes are collected to measure the concentrations of SP using ELISA (Enzyme-Linked Immunosorbent Assay). Basal SP levels (no stimulation, n=12) are compared against SP level following the stimulation with cold (n=12) and menthol (n=12) to see whether SP levels become substantially increased in the medium following the activation of TRPM8 receptors.

Yet another related embodiment contemplates identifying whether activation of TRPM8 receptors results in the release of SP from vagus afferent nerve terminals that are embedded in bronchopulmonary tissues. Experiments are performed using fresh bronchopulmonary tissues (containing vagus afferent terminals) to determine whether cold challenge (19° C. bath solution) and menthol stimulation (100 μM) result in the release of SP. The experiments and detection of SP release are similar to those using vagus ganglion neurons. If cold and menthol induce SP release, the effects of TRPM8 receptor antagonists (e.g. thio-BCTC, 10 μM; BCTC 10 μM, capsazepine, 50 μM) are tested to assess whether they can block cold- and menthol-induced SP release.

### EXAMPLE 4

### Asthma Models

Asthma is characterized by airway hyper-reactivity (AHR), airway edema and inflammation, and bronchial constriction (Leong and Huston, 2001; Pauwels et al 2001). Murine models of asthma have facilitated the investigation of asthma pathogenesis. However each model has specific limitations in the interpretation of the asthma diathesis and significant controversy exists about the development of true “asthma” as opposed to “allergic bronchopulmonary inflammation” since most models are associated with an allergic alveolitis or hypersensitivity pneumonitis (Kumar and Foster, 2002).

According to the subject invention, one asthma model provides asthma induction by sensitization and challenge of mice with ovalbumin (also referred to herein as the Ovalbumin model). This is a conventional animal model of asthma that has been well established and often used in asthma research.

Another animal model provides asthma induction by allergen sensitization and challenge of mice with cockroach extract antigen (also referred to herein as the Cockroach antigen model). This asthma animal model has been developed more recently and is relevant to asthma caused by indoor allergens. Cockroach antigen model has been well established in the laboratory of Dr. Kamal Mohammed, a faculty member in the University of Florida's College of Medicine.

### 1. Ovalbumin Model

The classic ovalbumin model involves development of an immunologic response but is not a model of a true environmental allergen exposure (Kumar et al 2000; Foster and Ming, 2000). The sensitization and challenge of mice is achieved with the method described previously. In brief, C57BL mice (6 weeks of age, 10 mice) are injected intraperitoneally with 200 μg of ovalbumin and 1 mg of Imject Alum (Pierce Chemical, Rockford, Ill., USA). A second intraperitoneal injection of 20 μg of ovalbumin adsorbed to adjuvant is administered 10 days after the first injection. After an additional 10 days, the mice are exposed to an aerosol of 1% ovalbumin in water for 30 min at 1 day intervals for 3 days. Two days after the final exposure to the aerosolized ovalbumin, the animals are challenged with a nebula of 10% ovalbumin in water. Nebulization is performed in a plastic chamber connected to an ultrasonic nebulizer that allows entry of an ovalbumin aerosol.

### 2. Cockroach Antigen Model

A disproportionate increase in asthma prevalence in the inner city has been correlated with exposure to cockroach allergen. Up to 60% of children from urban areas who present with asthma demonstrate specific antibodies to cockroach allergen. The cockroach allergen model involves an environmental allergen (Campbell et al 1998; Lukacs et al 2002). In the subject model, the environmental allergen (cockroach allergen) and long term, intermittent exposure of naturally breathing animals to the allergen are conducted to develop a model that produces the classic features of chronic asthma. The animals demonstrate AHR, numerous eosinophils and Th2 lymphocytes, sub-epithelial edema and fibrosis. Airway remodeling with increases in vascularity and smooth muscle tissue is also present.

To produce this asthma animal model, C57BL/6 mice receive an intraperitoneal injection of 10 μg of cockroach allergin in IFA on day zero. Mice are then exposed to aerosolized cockroach allergen for 30 minutes on an every other day protocol with assessment of responses starting at 2 weeks. Exposure to the cockroach allergen is carried out in a whole body inhalation exposure system during which animals are held in a flow-through cage rack and cockroach allergen is aerosolized by the delivery of compressed air to a sidestream jet nebulizer and injected into the airstream entering the chamber. The aerosol generated by this nebulizer (Pari L. C.) has 80% particles with a diameter of <4 μm (and >40% particles with a diameter of >1 μm) which has a very high probability>90% of tracheobronchial deposition. This method of delivery of antigen has been validated and demonstrated to have a particle concentration that is maintained in the range of 10-20 mg/m³ by adjustment of the pressure regulator which controls air flow into the nebulizer. This approach is effective in controlled exposure: the co-efficient of variation of the mean aerosol concentration is <10% while the mean concentration over 18 exposures in a six-week experiment is 0.2 mg/m³.

### 3. Up-Regulation of TRPM8 Receptors on Asthma Animal Models

Inflammatory reactions in bronchopulmonary tissues of asthma animals are known to result in an increased level of nerve growth factor (NGF). According to the subject invention, NGF results in TRPM8 receptor up-regulated in cultured sensory neurons (FIGS. 8a and 8b). Thus, TRPM8 receptors may be up-regulated on bronchopulmonary vegus afferent neurons in asthma animals. To test this idea, immunostaining and Western Blot are performed to assess whether TRPM8 receptor expression becomes up-regulated in the asthma animal models. An up-regulation of TRPM8 receptors in inflammatory bronchopulmonary tissues will make animals more susceptible to cold.

### 4. Cold-Induced Asthmatic Reactions

Airway hyper-reactivity to cold air are measured in the above two animal models. Airway hyper-reactivity is measured (Warner et al., 2004) using a Buxco mouse plethysmograph, which is specifically designed for the low tidal volumes. As described in previous publications, mice are anesthetized with sodium pentobarbital and incubated via cannulation of the trachea with an 18-gauge metal tube, subsequently ventilated with a ventilator. Animal are placed in the plethysmograph and readings monitored by a computer. A second transducer is used to measure the pressure swings at the opening of the trachea tube (P_{aw}), referenced to the body box (i.e., pleural pressure), and to provide a measure of Tran pulmonary pressure (Pₜₚ=P_{aw}−P_{box}). The trachea transducer is calibrated at a constant pressure of 20 cm H₂O. Resistance is calibrated by the Buxco software by dividing the change in pressure (Pₜₚ) by the change in flow (F) (δPₜₚ/δF; units=cm H₂O/ml/second) at two time points from the volume curve, based on a percentage of the inspiratory volume. Baseline levels for each mouse are determined (5-minute readings) and then a cold challenge is given by ventilating a cold air (5° C.). The peak airway resistance is recorded as a measure of airway hyper-reactivity. According to the subject invention, as seen with asthma patients, administration of a cold challenge induces higher airway resistance in asthma animal models.

### 5. Prevention of Cold-Induced Asthmatic Reaction by TRPM8 Receptor Antagonists

A number of compounds have been shown to block TRPM8 receptors as reported by us (Suzuki et al., 2004) and other investigators (Valenzano et al., 2003; Behrendt et al, 2004). These compounds are BCTC(N-(4-Tertiarybutylphenyl)-4-(3-cholorphyridin-2-yl)tetrahydropyrazine-1(2H)-carbox-amide, thio-BCTC, capsazepine, and 2-APB (2-aminoethoxydiphenyl borate). BCTC and thio-BCTC were synthesized by Purdue Pharma, L.P. Capsazepine and 2-APB are commercially available from a number of sources including Sigma. None of these compounds have been used clinically for respiratory diseases. In the previous in vitro studies, their antagonistic potencies (IC50) for TRPM8 receptors were capsazepine, 18±1.1 μM (Smart et al., 2001); thio-BCTC, 3.5±1.1 μM; BCTC 0.8±1.0 μM (Valenzano et al., 2003); and 2-APB, ˜50 μM. These results of in vitro experiments provide useful information for the in vivo experiments described below.

For the asthma animals for which cold triggers asthmatic reaction (airway hyper-reactivity to cold air, see above), the subject invention can identify whether TRPM8 receptor antagonists can block cold-induced asthmatic reaction. In brief, asthmatic reaction is induced in mice of both Ovalbumin Model and Cockroach Antigen Model as described above. After finishing the test of cold-induced asthmatic reaction, animals are rested for 4 hours for a recovery from asthmatic reaction. Then, TRPM8 receptor antagonists mentioned herein are administered to these animals. This is followed by a second test of cold challenge to obtain airway resistance and to compare the results with the airway resistance before introduction of the antagonists.

In the above experimental embodiments, TRPM8 receptor antagonists are administered by two routes: intraperitoneal injection and inhalation. Intraperitoneal injections are performed 30 min before cold challenge. Inhalation of antagonists, administered though a nebulizer, are done 3 minutes before cold challenge. For the 3-min inhalation of TRPM8 antagonists, their concentrations at 20 μM for capsazepine, 10 μM for thio-BCTC, 1 μM for BCTC, and 50 μM for 2-APB are used. For intraperitoneal injections of TRPM8 receptor antagonists, the doses are 5 mg/kg for capsazepine, 2.5 mg/kg for thio-BCTC, 1 mg/kg for BCTC, and 10 mg for 2-APB. The concentrations and doses of these antagonists can be adjusted. According to the subject invention, TRPM8 antagonists antagonize cold-induced increases of airway resistance in asthmatic animals.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

I claim:

1. A method for treating respiratory diseases or conditions comprising:
a) diagnosing a patient with a respiratory disease or condition;
b) administering to the patient an effective amount of a compound that binds to the TRPM8 receptor to modulate TRPM8 receptor activity involved with cold-induced vagus nerve reflex.

2. The method of claim 1, wherein the patient is administered a compound selected from the group consisting of natural or synthetic peptides, ligands, blockers, agonists, antagonists, inhibitors, antibodies, polynucleotides, and modulators.

3. The method of claim 2, wherein the compound is an antagonist selected from the group consisting of N-(4-tertiarybutylphenyl)-4-(3-chloropyridin-2-yl)tetrahydropyrazine-1(2H)-carbox-amide (BCTC), thio-BCTC, caps azepine, and 2-aminoethoxydiphenylborane.

4. The method of claim 2, wherein the patient is administered a compound selected from the group consisting of menthol, eucalyptus oil, peppermint oil, icilin, cyclohexanol, 5-methyl-2-(1-methylethenyl), 6-isopropyl-9-methyl-1,4-dioxaspiro-(4,5)decane-2-methanol, (I)-menthone glycerol ketal, 5-methyl-2-(1-methyl ethyl)-cyclohexyl-2-hydroxypropionate, 1-menthyl lactate, acid/-menthyl ester, menthyl pyrrolidone carboxylate, eucalyptol, linalool, geraniol, hydroxycitronellal, WS-3, WS-23, N-(4-tertiarybutylphenyl)-4-(3-chloropyridin-2-yl)tetrahydropyrazine-1(2H)-carbox-amide (BCTC), thio-BCTC, capsazepine, and 2-aminoethoxydiphenylborane, and mixtures thereof.

5. The method of claim 4, wherein the patient is diagnosed with asthma and the patient is administered 2-aminoethoxydiphenylborane.

6. The method of claim 1, wherein said respiratory disease or condition is selected from the group consisting of asthmatic conditions, chronic pulmonary diseases, and other pulmonary diseases.

7. The method of claim 6, wherein the respiratory disease or condition is selected from the group consisting of allergen-induced asthma, exercise-induced asthma, pollution-induced asthma, cold-induced asthma, stress-induced asthma, viral-induced-asthma, chronic bronchitis with normal airflow, chronic bronchitis with airway obstruction, emphysema, asthmatic bronchitis, bullous disease, cystic fibrosis, pigeon fancier's disease, farmer's lung, acute respiratory distress syndrome, pneumonia, aspiration or inhalation injury, fat embolism in the lung, acidosis inflammation of the lung, acute pulmonary edema, acute mountain sickness, post-cardiac surgery, acute pulmonary hypertension, persistent pulmonary hypertension of the newborn, perinatal aspiration syndrome, hyaline membrane disease, acute pulmonary thromboembolism, heparin-protamine reactions, sepsis, status asthamticus and hypoxia.

8. A pharmaceutical composition comprising a compound that binds to a TRPM8 receptor that is involved with cold-induced vagus nerve reflex with a pharmaceutically acceptable carrier.

9. A method for treating a respiratory disease or condition comprising:
a) identifying a polynucleotide that encodes a TRPM8 receptor involved with cold-induced vagus nerve reflex;
b) diagnosing a patient with a respiratory disease or condition;
c) administering to the patient the identified polynucleotide.

10. The method of claim 9, wherein the polynucleotide is administered to the patient via an expression vector.

11. The method of claim 10, wherein the expression vector contains any one or combination of the following sequences: antisense sequences, DNA sequences, RNA sequences, or PNA sequences that control regions of the polynucleotide encoding a TRPM8 receptor.

12. A method for screening compounds for their ability to bind with and modulate TRPM8 receptor activity with regard to cold-induced vagus nerve reflex, said method comprising:
a) combining at least one TRPM8 receptor with at least one candidate compound under conditions that allow binding of the compound(s) to the TRPM8 receptor(s); and
b) detecting binding of the compound(s) to the TRPM8 receptor(s), wherein the binding results in modulation of TRPM8 receptor activity.

13. The method of claim 12, further comprising the step of identifying the candidate compound that binds with the TRPM8 receptor(s), wherein the compound is useful for the treatment of a respiratory disease or condition.

14. The method of claim 13, wherein the respiratory disease or condition is selected from the group consisting of asthmatic conditions, chronic pulmonary diseases, and other pulmonary diseases.

15. The method of claim 14, wherein the respiratory disease or condition is selected from the group consisting of allergen-induced asthma, exercise-induced asthma, pollution-induced asthma, cold-induced asthma, stress-induced asthma, viral-induced-asthma, chronic bronchitis with normal airflow, chronic bronchitis with airway obstruction, emphysema, asthmatic bronchitis, bullous disease, cystic fibrosis, pigeon fancier's disease, farmer's lung, acute respiratory distress syndrome, pneumonia, aspiration or inhalation injury, fat embolism in the lung, acidosis inflammation of the lung, acute pulmonary edema, acute mountain sickness, post-cardiac surgery, acute pulmonary hypertension, persistent pulmonary hypertension of the newborn, perinatal aspiration syndrome, hyaline membrane disease, acute pulmonary thromboembolism, heparin-protamine reactions, sepsis, status asthamticus and hypoxia.

16. The method of claim 12, wherein the candidate compound is any one or combination of the compounds selected from the group consisting of natural or synthetic peptides, ligands, blockers, agonists, antagonists, inhibitors, antibodies, polynucleotides, and modulators.

17. The method of claim 16, wherein the compound is an agonist selected from the group consisting of menthol, eucalyptus oil, peppermint oil, icilin, cyclohexanol, 5-methyl-2-(1-methylethenyl), 6-isopropyl-9-methyl-1,4-dioxaspiro-(4,5)decane-2-methanol, (I)-menthone glycerol ketal, 5-methyl-2-(1-methyl ethyl)-cyclohexyl-2-hydroxypropionate, 1-menthyl lactate, acid/-menthyl ester, menthyl pyrrolidone carboxylate, eucalyptol, linalool, geraniol, hydroxycitronellal, WS-3, WS-23, and mixtures thereof.

18. The method of claim 16, wherein the compound is an antagonist selected from the group consisting of N-(4-tertiarybutylphenyl)-4-(3-chloropyridin-2-yl)tetrahydropyrazine-1(2H)-carbox-amide (BCTC), thio-BCTC, capsazepine, and 2-aminoethoxydiphenylborane.

19. A method for identifying TRPM8 receptors that are involved in cold-induced vagus nerve reflex, said method comprising:
a) retrograde labeling vagus afferent nerves that innervate bronchopulmonary tissues;
b) harvesting and dissociating vagus afferent nerve ganglions; and
c) assessing vagus afferent nerve ganglion response to cold.
